(19) United States
(12) Patent Application Publication
(10) Pub. No.: US 2023/0034093 A1
(43) Pub. Date: Feb. 2, 2023

(54) Treatment Of Cognitive Impairment With Alpha-N-Acetylgalactosaminide Alpha-2,6-Sialyltransferase 5 (ST6GALNAC5) Inhibitors

(71) Applicant: Regeneron Pharmaceuticals, Inc., Tarrytown, NY (US)

(72) Inventors: Jonathan Marchini, Tarrytown, NY (US); Manuel Allen Revez Ferreira, Tarrytown, NY (US); Giovanni Coppola, Tarrytown, NY (US); Goncalo Abecasis, Tarrytown, NY (US); Aris Baras, Tarrytown, NY (US)

(21) Appl. No.: 17/854,759

(22) Filed: Jun. 30, 2022

Related U.S. Application Data
(60) Provisional application No. 63/217,937, filed on Jul. 2, 2021.

(51) Int. Cl. C12Q 1/6827; A61K 31/7105; C12N 15/113; C12N 9/22; C07K 14/47; C12Q 1/6883

(52) U.S. Cl. CPC C12Q 1/6827; A61K 31/7105; C12N 15/113; C12N 9/22; C07K 14/4703; C12Q 1/6883; C12N 2310/14; C12Q 2600/156

(57) ABSTRACT

The present disclosure provides methods of treating subjects having cognitive impairment or at risk of developing cognitive impairment, and methods of identifying subjects having an increased risk of developing cognitive impairment.

### REFERENCE TO SEQUENCE LISTING

This application includes a Sequence Listing submitted electronically as a text file named 18923808001SEQ, created on Jun. 25, 2022, with a size of 634 kilobytes. The Sequence Listing is incorporated herein by reference.

### FIELD

The present disclosure relates generally to the treatment of subjects having cognitive impairment with Alpha-N-Acetylgalactosaminide Alpha-2,6-Sialyltransferase 5 (ST6GALNAC5) inhibitors, and methods of identifying subjects having an increased risk of developing cognitive impairment.

### BACKGROUND

Cognitive impairment can be characterized by an individual's difficulty in remembering, learning new things, concentrating, or making decisions that affect their everyday life. Cognitive impairment ranges from mild to severe. With mild impairment, individuals may begin to notice changes in cognitive functions, but still be able to do their everyday activities. Severe levels of impairment can lead to losing the ability to understand the meaning or importance of something and the ability to talk or write, resulting in the inability to live independently. Cognitive impairment includes, but is not limited to, decreased myelin integrity, neurodegeneration, decreased grey/white matter contrast (GWC), and conversion of mild cognitive impairment to dementia.

GWC can be used across a diffuse set of brain regions to assess cognitive impairment. GWC is a measure of blurring between the boundaries of grey/white matter brain compartments and is thought to be an indicator of local variations in tissue integrity and myelin degradation, increasing water content in the white matter, or iron deposition (Uribe et al., Front. Aging Neurosci., 2018, 10, 89). Lower GWC is associated with aging and lower indices of cognition (Lewis et al., Neuroimage., 2018, 173, 341-350), as well as an increased rate of conversion from mild cognitive impairment to dementia (Jefferson et al., Brain Imaging Behav., 2015, 9, 141-8).

Alpha-N-Acetylgalactosaminide Alpha-2,6-Sialyltransferase 5 (ST6GALNAC5) is a gene that catalyzes the biosynthesis of ganglioside GD1alpha from GM1b in the brain (Okajima et al., J. Biol. Chem., 1999, 274, 30557-30562). The ST6GALNAC5 gene was previously identified as one of the genes that mediate breast cancer metastasis to the brain.

### SUMMARY

The present disclosure provides methods of treating a subject having cognitive impairment or at risk of developing cognitive impairment, the methods comprising administering an ST6GALNAC5 inhibitor to the subject.

The present disclosure also provides methods of treating a subject having decreased myelin integrity or at risk of developing decreased myelin integrity, the methods comprising administering an ST6GALNAC5 inhibitor to the subject.

The present disclosure also provides methods of treating a subject having neurodegeneration or at risk of developing neurodegeneration, the methods comprising administering an ST6GALNAC5 inhibitor to the subject.

The present disclosure also provides methods of treating a subject having decreased grey/white matter contrast (GWC) or at risk of developing decreased GWC, the methods comprising administering an ST6GALNAC5 inhibitor to the subject.

The present disclosure also provides methods of treating a subject undergoing conversion from mild cognitive impairment to dementia or at risk of conversion from mild cognitive impairment to dementia, the methods comprising administering an ST6GALNAC5 inhibitor to the subject.

The present disclosure also provides methods of treating a subject with a therapeutic agent that treats or inhibits cognitive impairment, wherein the subject has cognitive impairment or is at risk of developing cognitive impairment, the methods comprising the steps of: determining whether the subject has an ST6GALNAC5 missense variant nucleic acid molecule encoding an ST6GALNAC5 predicted loss-of-function polypeptide by: obtaining or having obtained a biological sample from the subject; and performing or having performed a sequence analysis on the biological sample to determine if the subject has a genotype comprising the ST6GALNAC5 missense variant nucleic acid molecule encoding the ST6GALNAC5 predicted loss-of-function polypeptide; and i) administering or continuing to administer the therapeutic agent that treats or inhibits cognitive impairment in a standard dosage amount to a subject that is ST6GALNAC5 reference, and/or administering an ST6GALNAC5 inhibitor to the subject; or ii) administering or continuing to administer the therapeutic agent that treats or inhibits cognitive impairment in an amount that is the same as or less than the standard dosage amount to a subject that is heterozygous for the ST6GALNAC5 missense variant nucleic acid molecule encoding the ST6GALNAC5 predicted loss-of-function polypeptide, and/or administering an ST6GALNAC5 inhibitor to the subject; wherein the presence of a genotype having the ST6GALNAC5 missense variant nucleic acid molecule encoding the ST6GALNAC5 predicted loss-of-function polypeptide indicates the subject has a decreased risk of developing cognitive impairment.

The present disclosure also provides methods of identifying a subject having an increased risk of developing cognitive impairment, the methods comprising: determining or having determined the presence or absence of an ST6GALNAC5 missense variant nucleic acid molecule encoding an ST6GALNAC5 predicted loss-of-function polypeptide in a biological sample obtained from the subject; wherein the subject has an increased risk of developing cognitive impairment when the subject is ST6GALNAC5 reference, and the subject has a decreased risk of developing cognitive impairment when the subject is heterozygous or homozygous for the ST6GALNAC5 missense variant nucleic acid molecule encoding an ST6GALNAC5 predicted loss-of-function polypeptide.

The present disclosure also provides methods of detecting an ST6GALNAC5 missense variant nucleic acid molecule, or the complement thereof, encoding an ST6GALNAC5 predicted loss-of-function polypeptide in a subject, the methods comprising assaying a biological sample obtained from the subject to determine whether a nucleic acid molecule in the biological sample is: i) a genomic nucleic acid molecule having a nucleotide sequence comprising a cytosine at a position corresponding to position 176,867 according to SEQ ID NO:2, or the complement thereof; ii) an mRNA molecule having a nucleotide sequence comprising: a cytosine at a position corresponding to position 600 according to SEQ ID NO:11, or the complement thereof; a cytosine at a position corresponding to position 639 according to SEQ ID NO:12, or the complement thereof; a cytosine at a position corresponding to position 562 according to SEQ ID NO:13, or the complement thereof; a cytosine at a position corresponding to position 515 according to SEQ ID NO:14, or the complement thereof; a cytosine at a position corresponding to position 579 according to SEQ ID NO:15, or the complement thereof; a cytosine at a position corresponding to position 416 according to SEQ ID NO:16, or the complement thereof; a cytosine at a position corresponding to position 639 according to SEQ ID NO:17, or the complement thereof; or a cytosine at a position corresponding to position 600 according to SEQ ID NO:18, or the complement thereof; or iii) a cDNA molecule produced from an mRNA molecule in the biological sample, wherein the cDNA molecule has a nucleotide sequence comprising: a cytosine at a position corresponding to position 600 according to SEQ ID NO:27, or the complement thereof; a cytosine at a position corresponding to position 639 according to SEQ ID NO:28, or the complement thereof; a cytosine at a position corresponding to position 562 according to SEQ ID NO:29, or the complement thereof; a cytosine at a position corresponding to position 515 according to SEQ ID NO:30, or the complement thereof; a cytosine at a position corresponding to position 579 according to SEQ ID NO:31, or the complement thereof; a cytosine at a position corresponding to position 416 according to SEQ ID NO:32, or the complement thereof; a cytosine at a position corresponding to position 639 according to SEQ ID NO:33, or the complement thereof; or a cytosine at a position corresponding to position 600 according to SEQ ID NO:34, or the complement thereof.

The present disclosure also provides therapeutic agents that treat, inhibit, or prevent cognitive impairment for use in the treatment or prevention of cognitive impairment (or for use in the preparation of a medicament for treating cognitive impairment) in a subject identified as having: i) a genomic nucleic acid molecule encoding an ST6GALNAC5 predicted loss-of-function polypeptide, or the complement thereof, wherein the genomic nucleic acid molecule has a nucleotide sequence comprising: a cytosine at a position corresponding to position 176,867 according to SEQ ID NO:2, or the complement thereof; ii) an mRNA molecule having a nucleotide sequence encoding an ST6GALNAC5 predicted loss-of-function polypeptide, wherein the nucleotide sequence comprises: a cytosine at a position corresponding to position 600 according to SEQ ID NO:11, or the complement thereof; a cytosine at a position corresponding to position 639 according to SEQ ID NO:12, or the complement thereof; a cytosine at a position corresponding to position 562 according to SEQ ID NO:13, or the complement thereof; a cytosine at a position corresponding to position 515 according to SEQ ID NO:14, or the complement thereof; a cytosine at a position corresponding to position 579 according to SEQ ID NO:15, or the complement thereof; a cytosine at a position corresponding to position 416 according to SEQ ID NO:16, or the complement thereof; a cytosine at a position corresponding to position 639 according to SEQ ID NO:17, or the complement thereof; or a cytosine at a position corresponding to position 600 according to SEQ ID NO:18, or the complement thereof; or iii) a cDNA molecule having a nucleotide sequence encoding an ST6GALNAC5 predicted loss-of-function polypeptide, wherein the nucleotide sequence comprises: a cytosine at a position corresponding to position 600 according to SEQ ID NO:27, or the complement thereof; a cytosine at a position corresponding to position 639 according to SEQ ID NO:28, or the complement thereof; a cytosine at a position corresponding to position 562 according to SEQ ID NO:29, or the complement thereof; a cytosine at a position corresponding to position 515 according to SEQ ID NO:30, or the complement thereof; a cytosine at a position corresponding to position 579 according to SEQ ID NO:31, or the complement thereof; a cytosine at a position corresponding to position 416 according to SEQ ID NO:32, or the complement thereof; a cytosine at a position corresponding to position 639 according to SEQ ID NO:33, or the complement thereof; or a cytosine at a position corresponding to position 600 according to SEQ ID NO:34, or the complement thereof.

The present disclosure also provides ST6GALNAC5 inhibitors for use in the treatment or prevention of cognitive impairment (or for use in the preparation of a medicament for treating or preventing cognitive impairment) in a subject that: a) is reference for an ST6GALNAC5 genomic nucleic acid molecule, an ST6GALNAC5 mRNA molecule, or an ST6GALNAC5 cDNA molecule; or b) is heterozygous for: i) a genomic nucleic acid molecule encoding an ST6GALNAC5 predicted loss-of-function polypeptide, or the complement thereof, wherein the genomic nucleic acid molecule has a nucleotide sequence comprising: a cytosine at a position corresponding to position 176,867 according to SEQ ID NO:2, or the complement thereof; ii) an mRNA molecule encoding an ST6GALNAC5 predicted loss-of-function polypeptide, or the complement thereof, wherein the mRNA molecule has a nucleotide sequence comprising: a cytosine at a position corresponding to position 600 according to SEQ ID NO:11, or the complement thereof; a cytosine at a position corresponding to position 639 according to SEQ ID NO:12, or the complement thereof; a cytosine at a position corresponding to position 562 according to SEQ ID NO:13, or the complement thereof; a cytosine at a position corresponding to position 515 according to SEQ ID NO:14, or the complement thereof; a cytosine at a position corresponding to position 579 according to SEQ ID NO:15, or the complement thereof; a cytosine at a position corresponding to position 416 according to SEQ ID NO:16, or the complement thereof; a cytosine at a position corresponding to position 639 according to SEQ ID NO:17, or the complement thereof; or a cytosine at a position corresponding to position 600 according to SEQ ID NO:18, or the complement thereof; or iii) a cDNA molecule encoding an ST6GALNAC5 predicted loss-of-function polypeptide, or the complement thereof, wherein the cDNA molecule has a nucleotide sequence comprising: a cytosine at a position corresponding to position 600 according to SEQ ID NO:27, or the complement thereof; a cytosine at a position corresponding to position 639 according to SEQ ID NO:28, or the complement thereof; a cytosine at a position corresponding to position 562 according to SEQ ID NO:29, or the complement thereof; a cytosine at a position corresponding to position 515 according to SEQ ID NO:30, or the complement thereof; a cytosine at a position corresponding to position 579 according to SEQ ID NO:31, or the complement thereof; a cytosine at a position corresponding to position 416 according to SEQ ID NO:32, or the complement thereof; a cytosine at a position corresponding to position 639 according to SEQ ID NO:33, or the complement thereof; or a cytosine at a position corresponding to position 600 according to SEQ ID NO:34, or the complement thereof.

### DESCRIPTION

Various terms relating to aspects of the present disclosure are used throughout the specification and claims. Such terms are to be given their ordinary meaning in the art, unless otherwise indicated. Other specifically defined terms are to be construed in a manner consistent with the definitions provided herein.

Unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is in no way intended that an order be inferred, in any respect. This holds for any possible non-expressed basis for interpretation, including matters of logic with respect to arrangement of steps or operational flow, plain meaning derived from grammatical organization or punctuation, or the number or type of aspects described in the specification.

As used herein, the singular forms “a,” “an” and “the” include plural referents unless the context clearly dictates otherwise.

As used herein, the term “about” means that the recited numerical value is approximate and small variations would not significantly affect the practice of the disclosed embodiments. Where a numerical value is used, unless indicated otherwise by the context, the term “about” means the numerical value can vary by ±10% and remain within the scope of the disclosed embodiments.

As used herein, the term “comprising” may be replaced with “consisting” or “consisting essentially of” in particular embodiments as desired.

As used herein, the term “isolated”, in regard to a nucleic acid molecule or a polypeptide, means that the nucleic acid molecule or polypeptide is in a condition other than its native environment, such as apart from blood and/or animal tissue. In some embodiments, an isolated nucleic acid molecule or polypeptide is substantially free of other nucleic acid molecules or other polypeptides, particularly other nucleic acid molecules or polypeptides of animal origin. In some embodiments, the nucleic acid molecule or polypeptide can be in a highly purified form, i.e., greater than 95% pure or greater than 99% pure. When used in this context, the term “isolated” does not exclude the presence of the same nucleic acid molecule or polypeptide in alternative physical forms, such as dimers or alternatively phosphorylated or derivatized forms.

As used herein, the terms “nucleic acid”, “nucleic acid molecule”, “nucleic acid sequence”, “polynucleotide”, or “oligonucleotide” can comprise a polymeric form of nucleotides of any length, can comprise DNA and/or RNA, and can be single-stranded, double-stranded, or multiple stranded. One strand of a nucleic acid also refers to its complement.

As used herein, the term “subject” includes any animal, including mammals. Mammals include, but are not limited to, farm animals (such as, for example, horse, cow, pig), companion animals (such as, for example, dog, cat), laboratory animals (such as, for example, mouse, rat, rabbits), and non-human primates (such as, for example, apes and monkeys). In some embodiments, the subject is a human. In some embodiments, the subject is a patient under the care of a physician.

A rare loss-of-function variant in the ST6GALNAC5 gene associated with a decreased risk of developing cognitive impairment in humans has been identified in accordance with the present disclosure. For example, a genetic alteration that changes the thymine at position 176,867 in the ST6GALNAC5 reference genomic nucleic acid molecule (see, SEQ ID NO:1) to a cytosine has been observed to indicate that the subject having such an alteration may have a decreased risk of developing cognitive impairment. It is believed that no variants of the ST6GALNAC5 gene or protein have any known association with cognitive impairment. Altogether, the genetic analyses described herein surprisingly indicate that the ST6GALNAC5 gene and, in particular, a variant in the ST6GALNAC5 gene, associates with a decreased risk of developing cognitive impairment. Therefore, subjects that are ST6GALNAC5 reference that have an increased risk of developing cognitive impairment, such as decreased myelin integrity, neurodegeneration, decreased GWC, and conversion of mild cognitive impairment to dementia, may be treated such that the cognitive impairment is prevented, the symptoms thereof are reduced, and/or development of symptoms is repressed. Accordingly, the present disclosure provides methods of leveraging the identification of such variants in subjects to identify or stratify risk in such subjects of developing cognitive impairment, such as decreased myelin integrity, neurodegeneration, decreased GWC, and conversion of mild cognitive impairment to dementia, or to diagnose subjects as having an increased risk of developing cognitive impairment, such as decreased myelin integrity, neurodegeneration, decreased GWC, and conversion of mild cognitive impairment to dementia, such that subjects at risk or subjects with active disease may be treated accordingly.

For purposes of the present disclosure, any particular subject can be categorized as having one of three ST6GALNAC5 genotypes: i) ST6GALNAC5 reference; ii) heterozygous for an ST6GALNAC5 missense variant nucleic acid molecule encoding an ST6GALNAC5 predicted loss-of-function polypeptide; or iii) homozygous for an ST6GALNAC5 missense variant nucleic acid molecule encoding an ST6GALNAC5 predicted loss-of-function polypeptide. A subject is ST6GALNAC5 reference when the subject does not have a copy of an ST6GALNAC5 missense variant nucleic acid molecule encoding an ST6GALNAC5 predicted loss-of-function polypeptide. A subject is heterozygous for an ST6GALNAC5 missense variant nucleic acid molecule encoding an ST6GALNAC5 predicted loss-of-function polypeptide when the subject has a single copy of an ST6GALNAC5 missense variant nucleic acid molecule encoding an ST6GALNAC5 predicted loss-of-function polypeptide. As used herein, an ST6GALNAC5 missense variant nucleic acid molecule is any ST6GALNAC5 nucleic acid molecule (such as, a genomic nucleic acid molecule, an mRNA molecule, or a cDNA molecule) encoding an ST6GALNAC5 polypeptide having a partial loss-of-function, a complete loss-of-function, a predicted partial loss-of-function, or a predicted complete loss-of-function. A subject who has an ST6GALNAC5 missense variant nucleic acid molecule encoding an ST6GALNAC5 predicted loss-of-function polypeptide having a partial loss-of-function (or predicted partial loss-of-function) is hypomorphic for ST6GALNAC5. The ST6GALNAC5 missense variant nucleic acid molecule encoding an ST6GALNAC5 predicted loss-of-function polypeptide can be any nucleic acid molecule encoding an ST6GALNAC5 Val135Ala, Val45Ala, Val45A1a*, or Val135A1a* polypeptide. A subject is homozygous for an ST6GALNAC5 missense variant nucleic acid molecule encoding an ST6GALNAC5 predicted loss-of-function polypeptide when the subject has two copies of an ST6GALNAC5 missense variant nucleic acid molecule encoding an ST6GALNAC5 predicted loss-of-function polypeptide.

For subjects that are genotyped or determined to be ST6GALNAC5 reference, such subjects have an increased risk of developing cognitive impairment, such as decreased myelin integrity, neurodegeneration, decreased GWC, and conversion of mild cognitive impairment to dementia. For subjects that are genotyped or determined to be either ST6GALNAC5 reference or heterozygous for an ST6GALNAC5 missense variant nucleic acid molecule encoding an ST6GALNAC5 predicted loss-of-function polypeptide, such subjects can be treated with an ST6GALNAC5 inhibitor.

In any of the embodiments described throughout the present disclosure, the ST6GALNAC5 missense variant nucleic acid molecule encoding an ST6GALNAC5 predicted loss-of-function polypeptide can be any ST6GALNAC5 nucleic acid molecule (such as, for example, genomic nucleic acid molecule, mRNA molecule, or cDNA molecule) encoding an ST6GALNAC5 polypeptide having a partial loss-of-function, a complete loss-of-function, a predicted partial loss-of-function, or a predicted complete loss-of-function. For example, the ST6GALNAC5 missense variant nucleic acid molecule can be any nucleic acid molecule encoding ST6GALNAC5 Val135Ala, Val45Ala, Val45A1a*, or Val135A1a*. In some embodiments, the ST6GALNAC5 missense variant nucleic acid molecule encodes ST6GALNAC5 Val135Ala. In some embodiments, the ST6GALNAC5 missense variant nucleic acid molecule encodes ST6GALNAC5 Val45Ala. In some embodiments, the ST6GALNAC5 missense variant nucleic acid molecule encodes ST6GALNAC5 Val45A1a*. In some embodiments, the ST6GALNAC5 missense variant nucleic acid molecule encodes ST6GALNAC5 Val135A1a*.

In any of the embodiments described throughout the present disclosure, the ST6GALNAC5 predicted loss-of-function polypeptide can be any ST6GALNAC5 polypeptide having a partial loss-of-function, a complete loss-of-function, a predicted partial loss-of-function, or a predicted complete loss-of-function. In any of the embodiments described throughout the present disclosure, the ST6GALNAC5 predicted loss-of-function polypeptide can be any of the ST6GALNAC5 polypeptides described herein including, for example, ST6GALNAC5 Val135Ala, Val45Ala, Val45A1a*, or Val135A1a*. In some embodiments, the ST6GALNAC5 predicted loss-of-function polypeptide is ST6GALNAC5 Val135Ala. In some embodiments, the ST6GALNAC5 predicted loss-of-function polypeptide is ST6GALNAC5 Val45Ala. In some embodiments, the ST6GALNAC5 predicted loss-of-function polypeptide is ST6GALNAC5 Val45A1a*. In some embodiments, the ST6GALNAC5 predicted loss-of-function polypeptide is ST6GALNAC5 Val135A1a*.

In any of the embodiments described throughout the present disclosure, the cognitive impairment is decreased myelin integrity, neurodegeneration, decreased GWC, or conversion of mild cognitive impairment to dementia. In any of the embodiments described throughout the present disclosure, the cognitive impairment is decreased myelin integrity. In any of the embodiments described throughout the present disclosure, the cognitive impairment is neurodegeneration. In any of the embodiments described throughout the present disclosure, the cognitive impairment is decreased GWC. In any of the embodiments described throughout the present disclosure, the cognitive impairment is conversion of mild cognitive impairment to dementia.

Symptoms of cognitive impairment include, but are not limited to, memory loss, frequently asking the same question or repeating the same story several times, not recognizing familiar people and places, having difficulty exercising judgment (such as knowing what to do in an emergency), changes in mood or behavior, vision problems, and difficulty planning and carrying out tasks.

The present disclosure provides methods of treating a subject having cognitive impairment or at risk of developing cognitive impairment, the methods comprising administering an ST6GALNAC5 inhibitor to the subject.

The present disclosure also provides methods of treating a subject having decreased myelin integrity or at risk of developing decreased myelin integrity, the methods comprising administering an ST6GALNAC5 inhibitor to the subject.

The present disclosure also provides methods of treating a subject having neurodegeneration or at risk of developing neurodegeneration, the methods comprising administering an ST6GALNAC5 inhibitor to the subject.

The present disclosure also provides methods of treating a subject having decreased GWC or at risk of developing decreased GWC, the methods comprising administering an ST6GALNAC5 inhibitor to the subject.

The present disclosure also provides methods of treating a subject having conversion of mild cognitive impairment to dementia or at risk of conversion from mild cognitive impairment to dementia, the methods comprising administering an ST6GALNAC5 inhibitor to the subject.

In some embodiments, the ST6GALNAC5 inhibitor comprises an inhibitory nucleic acid molecule. In some embodiments, the inhibitory nucleic acid molecule comprises an antisense molecule, a small interfering RNA (siRNA) molecule, or a short hairpin RNA (shRNA) molecule. In some embodiments, the inhibitory nucleic acid molecule comprises an antisense molecule. In some embodiments, the inhibitory nucleic acid molecule comprises an siRNA molecule. In some embodiments, the inhibitory nucleic acid molecule comprises an shRNA molecule. Such inhibitory nucleic acid molecules can be designed to target any region of an ST6GALNAC5 nucleic acid molecule, such as an mRNA molecule. In some embodiments, the inhibitory nucleic acid molecule hybridizes to a sequence within an ST6GALNAC5 genomic nucleic acid molecule or mRNA molecule and decreases expression of the ST6GALNAC5 polypeptide in a cell in the subject. In some embodiments, the ST6GALNAC5 inhibitor comprises an antisense molecule that hybridizes to an ST6GALNAC5 genomic nucleic acid molecule or mRNA molecule and decreases expression of the ST6GALNAC5 polypeptide in a cell in the subject. In some embodiments, the ST6GALNAC5 inhibitor comprises an siRNA that hybridizes to an ST6GALNAC5 genomic nucleic acid molecule or mRNA molecule and decreases expression of the ST6GALNAC5 polypeptide in a cell in the subject. In some embodiments, the ST6GALNAC5 inhibitor comprises an shRNA that hybridizes to an ST6GALNAC5 genomic nucleic acid molecule or mRNA molecule and decreases expression of the ST6GALNAC5 polypeptide in a cell in the subject.

The inhibitory nucleic acid molecules can comprise RNA, DNA, or both RNA and DNA. The inhibitory nucleic acid molecules can also be linked or fused to a heterologous nucleic acid sequence, such as in a vector, or a heterologous label. For example, the inhibitory nucleic acid molecules can be within a vector or as an exogenous donor sequence comprising the inhibitory nucleic acid molecule and a heterologous nucleic acid sequence. The inhibitory nucleic acid molecules can also be linked or fused to a heterologous label. The label can be directly detectable (such as, for example, fluorophore) or indirectly detectable (such as, for example, hapten, enzyme, or fluorophore quencher). Such labels can be detectable by spectroscopic, photochemical, biochemical, immunochemical, or chemical means. Such labels include, for example, radiolabels, pigments, dyes, chromogens, spin labels, and fluorescent labels. The label can also be, for example, a chemiluminescent substance; a metal-containing substance; or an enzyme, where there occurs an enzyme-dependent secondary generation of signal. The term “label” can also refer to a “tag” or hapten that can bind selectively to a conjugated molecule such that the conjugated molecule, when added subsequently along with a substrate, is used to generate a detectable signal. For example, biotin can be used as a tag along with an avidin or streptavidin conjugate of horseradish peroxidate (HRP) to bind to the tag, and examined using a calorimetric substrate (such as, for example, tetramethylbenzidine (TMB)) or a fluorogenic substrate to detect the presence of HRP. Exemplary labels that can be used as tags to facilitate purification include, but are not limited to, myc, HA, FLAG or 3×FLAG, 6×His or polyhistidine, glutathione-S-transferase (GST), maltose binding protein, an epitope tag, or the Fc portion of immunoglobulin. Numerous labels include, for example, particles, fluorophores, haptens, enzymes and their calorimetric, fluorogenic and chemiluminescent substrates and other labels.

The inhibitory nucleic acid molecules can comprise, for example, nucleotides or non-natural or modified nucleotides, such as nucleotide analogs or nucleotide substitutes. Such nucleotides include a nucleotide that contains a modified base, sugar, or phosphate group, or that incorporates a non-natural moiety in its structure. Examples of non-natural nucleotides include, but are not limited to, dideoxynucleotides, biotinylated, aminated, deaminated, alkylated, benzylated, and fluorophor-labeled nucleotides.

The inhibitory nucleic acid molecules can also comprise one or more nucleotide analogs or substitutions. A nucleotide analog is a nucleotide which contains a modification to either the base, sugar, or phosphate moieties. Modifications to the base moiety include, but are not limited to, natural and synthetic modifications of A, C, G, and T/U, as well as different purine or pyrimidine bases such as, for example, pseudouridine, uracil-5-yl, hypoxanthin-9-yl (I), and 2-aminoadenin-9-yl. Modified bases include, but are not limited to, 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl uracil and cytosine, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo (such as, for example, 5-bromo), 5-trifluoromethyl and other 5-substituted uracils and cytosines, 7-methylguanine, 7-methyladenine, 8-azaguanine, 8-azaadenine, 7-deazaguanine, 7-deazaadenine, 3-deazaguanine, and 3-deazaadenine.

Nucleotide analogs can also include modifications of the sugar moiety. Modifications to the sugar moiety include, but are not limited to, natural modifications of the ribose and deoxy ribose as well as synthetic modifications. Sugar modifications include, but are not limited to, the following modifications at the 2′ position: OH; F; O-, S-, or N-alkyl; O-, S-, or N-alkenyl; O-, S- or N-alkynyl; or O-alkyl-O-alkyl, wherein the alkyl, alkenyl, and alkynyl may be substituted or unsubstituted C₁₋₁₀alkyl or C₂₋₁₀alkenyl, and C₂₋₁₀alkynyl. Exemplary 2′ sugar modifications also include, but are not limited to, —O[(CH₂)ₙO]ₘCH₃, —O(CH₂)ₙOCH₃, —O(CH₂)ₙNH₂, —O(CH₂)ₙCH₃, —O(CH₂)ₙ—ONH₂, and —O(CH₂)ₙON[(CH₂)ₙCH₃)]₂, where n and m, independently, are from 1 to about 10. Other modifications at the 2′ position include, but are not limited to, C₁₋₁₀alkyl, substituted lower alkyl, alkaryl, aralkyl, O-alkaryl or O-aralkyl, SH, SCH₃, OCN, Cl, Br, CN, CF₃, OCF₃, SOCH₃, SO₂CH₃, ONO₂, NO₂, N₃, NH₂, heterocycloalkyl, heterocycloalkaryl, aminoalkylamino, polyalkylamino, substituted silyl, an RNA cleaving group, a reporter group, an intercalator, a group for improving the pharmacokinetic properties of an oligonucleotide, or a group for improving the pharmacodynamic properties of an oligonucleotide, and other substituents having similar properties. Similar modifications may also be made at other positions on the sugar, particularly the 3′ position of the sugar on the 3′ terminal nucleotide or in 2′-5′ linked oligonucleotides and the 5′ position of 5′ terminal nucleotide. Modified sugars can also include those that contain modifications at the bridging ring oxygen, such as CH₂ and S. Nucleotide sugar analogs can also have sugar mimetics, such as cyclobutyl moieties in place of the pentofuranosyl sugar.

Nucleotide analogs can also be modified at the phosphate moiety. Modified phosphate moieties include, but are not limited to, those that can be modified so that the linkage between two nucleotides contains a phosphorothioate, chiral phosphorothioate, phosphorodithioate, phosphotriester, aminoalkylphosphotriester, methyl and other alkyl phosphonates including 3′-alkylene phosphonate and chiral phosphonates, phosphinates, phosphoramidates including 3′-amino phosphoramidate and aminoalkylphosphoramidates, thionophosphoramidates, thionoalkylphosphonates, thionoalkylphosphotriesters, and boranophosphates. These phosphate or modified phosphate linkage between two nucleotides can be through a 3′-5′ linkage or a 2′-5′ linkage, and the linkage can contain inverted polarity such as 3′-5′ to 5′-3′ or 2′-5′ to 5′-2′. Various salts, mixed salts, and free acid forms are also included. Nucleotide substitutes also include peptide nucleic acids (PNAs).

In some embodiments, the antisense nucleic acid molecules are gapmers, whereby the first one to seven nucleotides at the 5′ and 3′ ends each have 2′-methoxyethyl (2′-MOE) modifications. In some embodiments, the first five nucleotides at the 5′ and 3′ ends each have 2′-MOE modifications. In some embodiments, the first one to seven nucleotides at the 5′ and 3′ ends are RNA nucleotides. In some embodiments, the first five nucleotides at the 5′ and 3′ ends are RNA nucleotides. In some embodiments, each of the backbone linkages between the nucleotides is a phosphorothioate linkage.

In some embodiments, the siRNA molecules have termini modifications. In some embodiments, the 5′ end of the antisense strand is phosphorylated. In some embodiments, 5′-phosphate analogs that cannot be hydrolyzed, such as 5′-(E)-vinyl-phosphonate are used.

In some embodiments, the siRNA molecules have backbone modifications. In some embodiments, the modified phosphodiester groups that link consecutive ribose nucleosides have been shown to enhance the stability and in vivo bioavailability of siRNAs The non-ester groups (—OH, ═O) of the phosphodiester linkage can be replaced with sulfur, boron, or acetate to give phosphorothioate, boranophosphate, and phosphonoacetate linkages. In addition, substituting the phosphodiester group with a phosphotriester can facilitate cellular uptake of siRNAs and retention on serum components by eliminating their negative charge. In some embodiments, the siRNA molecules have sugar modifications. In some embodiments, the sugars are deprotonated (reaction catalyzed by exo- and endonucleases) whereby the 2′-hydroxyl can act as a nucleophile and attack the adjacent phosphorous in the phosphodiester bond. Such alternatives include 2′-O-methyl, 2′-O-methoxyethyl, and 2′-fluoro modifications.

In some embodiments, the siRNA molecules have base modifications. In some embodiments, the bases can be substituted with modified bases such as pseudouridine, 5′-methylcytidine, N6-methyladenosine, inosine, and N7-methylguanosine.

In some embodiments, the siRNA molecules are conjugated to lipids. Lipids can be conjugated to the 5′ or 3′ termini of siRNA to improve their in vivo bioavailability by allowing them to associate with serum lipoproteins. Representative lipids include, but are not limited to, cholesterol and vitamin E, and fatty acids, such as palmitate and tocopherol.

In some embodiments, a representative siRNA has the following formula:

Sense: mN*mN*/i2FN/mN/i2FN/mN/i2FN/mN/i2FN/mN/i2FN/mN/i2FN/mN/i2FN/mN/i2FN/*mN*/32FN/

### Antisense: /52FN/*/i2FN/*mN/i2FN/mN/i2FN/mN/i2FN/mN/i2FN/mN/i2FN/mN/i2FN/mN/i2FN/mN/i2FN/mN*N*N

wherein: “N” is the base; “2F” is a 2′-F modification; “m” is a 2′-O-methyl modification, “I” is an internal base; and “*” is a phosphorothioate backbone linkage.

The present disclosure also provides vectors comprising any one or more of the inhibitory nucleic acid molecules. In some embodiments, the vectors comprise any one or more of the inhibitory nucleic acid molecules and a heterologous nucleic acid. The vectors can be viral or nonviral vectors capable of transporting a nucleic acid molecule. In some embodiments, the vector is a plasmid or cosmid (such as, for example, a circular double-stranded DNA into which additional DNA segments can be ligated). In some embodiments, the vector is a viral vector, wherein additional DNA segments can be ligated into the viral genome. Expression vectors include, but are not limited to, plasmids, cosmids, retroviruses, adenoviruses, adeno-associated viruses (AAV), plant viruses such as cauliflower mosaic virus and tobacco mosaic virus, yeast artificial chromosomes (YACs), Epstein-Barr (EBV)-derived episomes, and other expression vectors known in the art.

The present disclosure also provides compositions comprising any one or more of the inhibitory nucleic acid molecules. In some embodiments, the composition is a pharmaceutical composition. In some embodiments, the compositions comprise a carrier and/or excipient. Examples of carriers include, but are not limited to, poly(lactic acid) (PLA) microspheres, poly(D,L-lactic-coglycolic-acid) (PLGA) microspheres, liposomes, micelles, inverse micelles, lipid cochleates, and lipid microtubules. A carrier may comprise a buffered salt solution such as PBS, HBSS, etc.

In some embodiments, the ST6GALNAC5 inhibitor comprises a nuclease agent that induces one or more nicks or double-strand breaks at a recognition sequence(s) or a DNA-binding protein that binds to a recognition sequence within an ST6GALNAC5 genomic nucleic acid molecule. The recognition sequence can be located within a coding region of the ST6GALNAC5 gene, or within regulatory regions that influence the expression of the gene. A recognition sequence of the DNA-binding protein or nuclease agent can be located in an intron, an exon, a promoter, an enhancer, a regulatory region, or any non-protein coding region. The recognition sequence can include or be proximate to the start codon of the ST6GALNAC5 gene. For example, the recognition sequence can be located about 10, about 20, about 30, about 40, about 50, about 100, about 200, about 300, about 400, about 500, or about 1,000 nucleotides from the start codon. As another example, two or more nuclease agents can be used, each targeting a nuclease recognition sequence including or proximate to the start codon. As another example, two nuclease agents can be used, one targeting a nuclease recognition sequence including or proximate to the start codon, and one targeting a nuclease recognition sequence including or proximate to the stop codon, wherein cleavage by the nuclease agents can result in deletion of the coding region between the two nuclease recognition sequences. Any nuclease agent that induces a nick or double-strand break into a desired recognition sequence can be used in the methods and compositions disclosed herein. Any DNA-binding protein that binds to a desired recognition sequence can be used in the methods and compositions disclosed herein.

Suitable nuclease agents and DNA-binding proteins for use herein include, but are not limited to, zinc finger protein or zinc finger nuclease (ZFN) pair, Transcription Activator-Like Effector (TALE) protein or Transcription Activator-Like Effector Nuclease (TALEN), or

Clustered Regularly Interspersed Short Palindromic Repeats (CRISPR)/CRISPR-associated (Cas) systems. The length of the recognition sequence can vary, and includes, for example, recognition sequences that are about 30-36 bp for a zinc finger protein or ZFN pair, about 15-18 bp for each ZFN, about 36 bp for a TALE protein or TALEN, and about 20 bp for a CRISPR/Cas guide RNA.

In some embodiments, CRISPR/Cas systems can be used to modify an ST6GALNAC5 genomic nucleic acid molecule within a cell. The methods and compositions disclosed herein can employ CRISPR-Cas systems by utilizing CRISPR complexes (comprising a guide RNA (gRNA) complexed with a Cas protein) for site-directed cleavage of ST6GALNAC5 nucleic acid molecules.

Cas proteins generally comprise at least one RNA recognition or binding domain that can interact with gRNAs. Cas proteins can also comprise nuclease domains (such as, for example, DNase or RNase domains), DNA binding domains, helicase domains, protein-protein interaction domains, dimerization domains, and other domains. Suitable Cas proteins include, for example, a wild type Cas9 protein and a wild type Cpf1 protein (such as, for example, FnCpf1). A Cas protein can have full cleavage activity to create a double-strand break in an ST6GALNAC5 genomic nucleic acid molecule or it can be a nickase that creates a single-strand break in an ST6GALNAC5 genomic nucleic acid molecule. Additional examples of Cas proteins include, but are not limited to, Cas1, Cas1B, Cast, Cas3, Cas4, Cas5, Cas5e (CasD), Cas6, Cas6e, Cas6f, Cas7, Cas8a1, Cas8a2, Cas8b, Cas8c, Cas9 (Csn1 or Csx12), Cas10, Cas10d, CasF, CasG, CasH, Csy1, Csy2, Csy3, Cse1 (CasA), Cse2 (Cas6), Cse3 (CasE), Cse4 (CasC), Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, and Cu1966, and homologs or modified versions thereof. Cas proteins can also be operably linked to heterologous polypeptides as fusion proteins. For example, a Cas protein can be fused to a cleavage domain, an epigenetic modification domain, a transcriptional activation domain, or a transcriptional repressor domain. Cas proteins can be provided in any form. For example, a Cas protein can be provided in the form of a protein, such as a Cas protein complexed with a gRNA. Alternately, a Cas protein can be provided in the form of a nucleic acid molecule encoding the Cas protein, such as an RNA or DNA.

In some embodiments, targeted genetic modifications of an ST6GALNAC5 genomic nucleic acid molecules can be generated by contacting a cell with a Cas protein and one or more gRNAs that hybridize to one or more gRNA recognition sequences within a target genomic locus in the ST6GALNAC5 genomic nucleic acid molecule. For example, a gRNA recognition sequence can be located within a region of SEQ ID NO:1. The gRNA recognition sequence can also include or be proximate to a position corresponding to position 176,867 according to SEQ ID NO:1. For example, the gRNA recognition sequence can be located from about 1000, from about 500, from about 400, from about 300, from about 200, from about 100, from about 50, from about 45, from about 40, from about 35, from about 30, from about 25, from about 20, from about 15, from about 10, or from about 5 nucleotides of a position corresponding to position 176,867 according to SEQ ID NO:1. The gRNA recognition sequence can include or be proximate to the start codon of an ST6GALNAC5 genomic nucleic acid molecule or the stop codon of an ST6GALNAC5 genomic nucleic acid molecule. For example, the gRNA recognition sequence can be located from about 10, from about 20, from about 30, from about 40, from about 50, from about 100, from about 200, from about 300, from about 400, from about 500, or from about 1,000 nucleotides of the start codon or the stop codon.

The gRNA recognition sequences within a target genomic locus in an ST6GALNAC5 genomic nucleic acid molecule are located near a Protospacer Adjacent Motif (PAM) sequence, which is a 2-6 base pair DNA sequence immediately following the DNA sequence targeted by the Cas9 nuclease. The canonical PAM is the sequence 5′-NGG-3′ where “N” is any nucleobase followed by two guanine (“G”) nucleobases. gRNAs can transport Cas9 to anywhere in the genome for gene editing, but no editing can occur at any site other than one at which Cas9 recognizes PAM. In addition, 5′-NGA-3′ can be a highly efficient non-canonical PAM for human cells. Generally, the PAM is about 2 to about 6 nucleotides downstream of the DNA sequence targeted by the gRNA. The PAM can flank the gRNA recognition sequence. In some embodiments, the gRNA recognition sequence can be flanked on the 3′ end by the PAM. In some embodiments, the gRNA recognition sequence can be flanked on the 5′ end by the PAM. For example, the cleavage site of Cas proteins can be about 1 to about 10 base pairs, about 2 to about 5 base pairs, or 3 base pairs upstream or downstream of the PAM sequence. In some embodiments (such as when Cas9 from S. pyogenes or a closely related Cas9 is used), the PAM sequence of the non-complementary strand can be 5′-NGG-3′, where N is any DNA nucleotide and is immediately 3′ of the gRNA recognition sequence of the non-complementary strand of the target DNA. As such, the PAM sequence of the complementary strand would be 5′-CCN-3′, where N is any DNA nucleotide and is immediately 5′ of the gRNA recognition sequence of the complementary strand of the target DNA.

A gRNA is an RNA molecule that binds to a Cas protein and targets the Cas protein to a specific location within an ST6GALNAC5 genomic nucleic acid molecule. An exemplary gRNA is a gRNA effective to direct a Cas enzyme to bind to or cleave an ST6GALNAC5 genomic nucleic acid molecule, wherein the gRNA comprises a DNA-targeting segment that hybridizes to a gRNA recognition sequence within the ST6GALNAC5 genomic nucleic acid molecule that includes or is proximate to a position corresponding to position 176,867 according to SEQ ID NO:1. For example, a gRNA can be selected such that it hybridizes to a gRNA recognition sequence that is located about 5, about 10, about 15, about 20, about 25, about 30, about 35, about 40, about 45, about 50, about 100, about 200, about 300, about 400, about 500, or about 1,000 nucleotides from a position corresponding to position 176,867 according to SEQ ID NO:1. Other exemplary gRNAs comprise a DNA-targeting segment that hybridizes to a gRNA recognition sequence present within an ST6GALNAC5 genomic nucleic acid molecule that includes or is proximate to the start codon or the stop codon. For example, a gRNA can be selected such that it hybridizes to a gRNA recognition sequence that is located about 5, about 10, about 15, about 20, about 25, about 30, about 35, about 40, about 45, about 50, about 100, about 200, about 300, about 400, about 500, or about 1,000 nucleotides of the start codon or located about 5, about 10, about 15, about 20, about 25, about 30, about 35, about 40, about 45, about 50, about 100, about 200, about 300, about 400, about 500, or about 1,000 nucleotides of the stop codon. Suitable gRNAs can comprise from about 17 to about 25 nucleotides, from about 17 to about 23 nucleotides, from about 18 to about 22 nucleotides, or from about 19 to about 21 nucleotides. In some embodiments, the gRNAs can comprise 20 nucleotides.

Examples of suitable gRNA recognition sequences located within the ST6GALNAC5 reference gene are set forth in Table 1 as SEQ ID NOs:43-62.

TABLE 1
Guide RNA Recognition Sequences Within ST6GALNAC5

| Strand | gRNA Recognition Sequence | SEQ ID NO: |
|---|---|---|
| − | CTTGTGGCGAGTAATCATGA | 43 |
| + | ATGAATGACGCCCCCACACG | 44 |
| + | GGACGGATACCTCGGAGTGG | 45 |
| + | CGCGGCTATGGGCGTGACGT | 46 |
| − | GTGTGGGGGCGTCATTCATG | 47 |
| − | GCTGTACACTAGCAACAAGC | 48 |
| + | GGGGCCCCAGCAGCTACATG | 49 |
| − | CCTTGTGGTCCGCCACTCCG | 50 |
| + | CCTCGGAGTGGCGGACCACA | 51 |
| − | ACTCCGAGGTATCCGTCCAG | 52 |
| + | TGGGCAATCGCACCAGCCTG | 53 |
| + | AGCAGCTACATGCGGCGGGA | 54 |
| − | AGGTATCCGTCCAGTGGCCG | 55 |
| − | GTCACGCCCATAGCCGCGTG | 56 |
| − | CACGCCCATAGCCGCGTGTG | 57 |
| + | TCGCGCATTCCAGCATCCAG | 58 |
| − | ACGCCCATAGCCGCGTGTGG | 59 |
| − | GCGGGCATGCTGTCACCTTG | 60 |
| + | CATCTGCTGCACAGTCGGCA | 61 |
| + | CAGCAGCAGGCGTCGGCCAC | 62 |

The Cas protein and the gRNA form a complex, and the Cas protein cleaves the target ST6GALNAC5 genomic nucleic acid molecule. The Cas protein can cleave the nucleic acid molecule at a site within or outside of the nucleic acid sequence present in the target ST6GALNAC5 genomic nucleic acid molecule to which the DNA-targeting segment of a gRNA will bind. For example, formation of a CRISPR complex (comprising a gRNA hybridized to a gRNA recognition sequence and complexed with a Cas protein) can result in cleavage of one or both strands in or near (such as, for example, within 1, 2, 3, 4, 5, 6, 7, 8, 9, 10, 20, 50, or more base pairs from) the nucleic acid sequence present in the ST6GALNAC5 genomic nucleic acid molecule to which a DNA-targeting segment of a gRNA will bind.

Such methods can result, for example, in an ST6GALNAC5 genomic nucleic acid molecule in which a region of SEQ ID NO:1 is disrupted, the start codon is disrupted, the stop codon is disrupted, or the coding sequence is disrupted or deleted. Optionally, the cell can be further contacted with one or more additional gRNAs that hybridize to additional gRNA recognition sequences within the target genomic locus in the ST6GALNAC5 genomic nucleic acid molecule. By contacting the cell with one or more additional gRNAs (such as, for example, a second gRNA that hybridizes to a second gRNA recognition sequence), cleavage by the Cas protein can create two or more double-strand breaks or two or more single-strand breaks.

In some embodiments, the ST6GALNAC5 inhibitor comprises a small molecule. In some embodiments, the ST6GALNAC5 inhibitor is myricetin.

In some embodiments, the methods of treatment further comprise detecting the presence or absence of an ST6GALNAC5 missense variant nucleic acid molecule encoding an ST6GALNAC5 predicted loss-of-function polypeptide in a biological sample obtained from the subject. As used throughout the present disclosure, “an ST6GALNAC5 missense variant nucleic acid molecule” is any ST6GALNAC5 nucleic acid molecule (such as, for example, genomic nucleic acid molecule, mRNA molecule, or cDNA molecule) encoding an ST6GALNAC5 polypeptide having a partial loss-of-function, a complete loss-of-function, a predicted partial loss-of-function, or a predicted complete loss-of-function.

The present disclosure also provides methods of treating a subject with a therapeutic agent that treats or inhibits cognitive impairment. In some embodiments, the subject has cognitive impairment. In some embodiments, the methods comprise determining whether the subject has an ST6GALNAC5 missense variant nucleic acid molecule encoding an ST6GALNAC5 predicted loss-of-function polypeptide by obtaining or having obtained a biological sample from the subject, and performing or having performed a sequence analysis on the biological sample to determine if the subject has a genotype comprising the ST6GALNAC5 missense variant nucleic acid molecule. When the subject is ST6GALNAC5 reference, the therapeutic agent that treats or inhibits cognitive impairment is administered or continued to be administered to the subject in a standard dosage amount, and/or an ST6GALNAC5 inhibitor is administered to the subject. When the subject is heterozygous for an ST6GALNAC5 missense variant nucleic acid molecule, the therapeutic agent that treats or inhibits cognitive impairment is administered or continued to be administered to the subject in an amount that is the same as or less than a standard dosage amount, and/or an ST6GALNAC5 inhibitor is administered to the subject. The presence of a genotype having an ST6GALNAC5 missense variant nucleic acid molecule encoding an ST6GALNAC5 predicted loss-of-function polypeptide indicates the subject has a decreased risk of developing cognitive impairment. In some embodiments, the subject is ST6GALNAC5 reference. In some embodiments, the subject is heterozygous for an ST6GALNAC5 missense variant nucleic acid molecule encoding an ST6GALNAC5 predicted loss-of-function polypeptide.

For subjects that are genotyped or determined to be either ST6GALNAC5 reference or heterozygous for an ST6GALNAC5 missense variant nucleic acid molecule encoding an ST6GALNAC5 predicted loss-of-function polypeptide, such subjects can be treated with an ST6GALNAC5 inhibitor, as described herein.

Detecting the presence or absence of an ST6GALNAC5 missense variant nucleic acid molecule encoding an ST6GALNAC5 predicted loss-of-function polypeptide in a biological sample from a subject and/or determining whether a subject has an ST6GALNAC5 missense variant nucleic acid molecule encoding an ST6GALNAC5 predicted loss-of-function polypeptide can be carried out by any of the methods described herein. In some embodiments, these methods can be carried out in vitro. In some embodiments, these methods can be carried out in situ. In some embodiments, these methods can be carried out in vivo. In any of these embodiments, the ST6GALNAC5 missense variant nucleic acid molecule encoding an ST6GALNAC5 predicted loss-of-function polypeptide can be present within a cell obtained from the subject.

In some embodiments, when the subject is ST6GALNAC5 reference, the subject is also administered a therapeutic agent that treats or inhibits cognitive impairment in a standard dosage amount. In some embodiments, when the subject is heterozygous for an ST6GALNAC5 missense variant nucleic acid molecule encoding an ST6GALNAC5 predicted loss-of-function polypeptide, the subject is also administered a therapeutic agent that treats or inhibits cognitive impairment in a dosage amount that is the same as or less than a standard dosage amount.

In some embodiments, the treatment methods further comprise detecting the presence or absence of an ST6GALNAC5 predicted loss-of-function polypeptide in a biological sample from the subject. In some embodiments, when the subject does not have an ST6GALNAC5 predicted loss-of-function polypeptide, the subject is also administered a therapeutic agent that treats or inhibits cognitive impairment in a standard dosage amount. In some embodiments, when the subject has an ST6GALNAC5 predicted loss-of-function polypeptide, the subject is also administered a therapeutic agent that treats or inhibits cognitive impairment in a dosage amount that is the same as or less than a standard dosage amount.

The present disclosure also provides methods of treating a subject with a therapeutic agent that treats or prevents cognitive impairment. In some embodiments, the subject has cognitive impairment. In some embodiments, the subject is at risk of developing cognitive impairment. In some embodiments, the method comprises determining whether the subject has an ST6GALNAC5 predicted loss-of-function polypeptide by obtaining or having obtained a biological sample from the subject, and performing or having performed an assay on the biological sample to determine if the subject has an ST6GALNAC5 predicted loss-of-function polypeptide. When the subject does not have an ST6GALNAC5 predicted loss-of-function polypeptide, the therapeutic agent that treats or inhibits cognitive impairment is administered or continued to be administered to the subject in a standard dosage amount, and/or an ST6GALNAC5 inhibitor is administered to the subject. When the subject has an ST6GALNAC5 predicted loss-of-function polypeptide, the therapeutic agent that treats or inhibits cognitive impairment is administered or continued to be administered to the subject in an amount that is the same as or less than a standard dosage amount, and/or an ST6GALNAC5 inhibitor is administered to the subject. The presence of an ST6GALNAC5 predicted loss-of-function polypeptide indicates the subject has a decreased risk of developing cognitive impairment. In some embodiments, the subject has an ST6GALNAC5 predicted loss-of-function polypeptide. In some embodiments, the subject does not have an ST6GALNAC5 predicted loss-of-function polypeptide.

Detecting the presence or absence of an ST6GALNAC5 predicted loss-of-function polypeptide in a biological sample from a subject and/or determining whether a subject has an ST6GALNAC5 predicted loss-of-function polypeptide can be carried out by any of the methods described herein. In some embodiments, these methods can be carried out in vitro. In some embodiments, these methods can be carried out in situ. In some embodiments, these methods can be carried out in vivo. In any of these embodiments, the ST6GALNAC5 predicted loss-of-function polypeptide can be present within a cell obtained from the subject.

Examples of therapeutic agents that treat or prevent cognitive impairment include, but are not limited to: a cholinesterase inhibitor, vitamin E, and ginkgo. In some embodiments, the therapeutic agent that treats or inhibits cognitive impairment is a cholinesterase inhibitor. In some embodiments, the therapeutic agent that treats or inhibits cognitive impairment is vitamin E. In some embodiments, the therapeutic agent that treats or inhibits cognitive impairment is ginkgo. In some embodiments, the cholinesterase inhibitor is ARICEPT® or ARICEPT® ODT (donepezil), COGNEX® (tacrine), EXELON® or EXELON® Patch (rivastigmine), RAZADYNE® (galantamine), NAMZARIC® (memantine/donepezil), MYTELASE® (ambenonium), or BLOXIVERZ® (neostigmine). In some embodiments, the cholinesterase inhibitor is donepezil, tacrine, rivastigmine, galantamine, memantine/donepezil, ambenonium, or neostigmine. In some embodiments, the cholinesterase inhibitor is donepezil. In some embodiments, the cholinesterase inhibitor is tacrine. In some embodiments, the cholinesterase inhibitor is rivastigmine. In some embodiments, the cholinesterase inhibitor is galantamine. In some embodiments, the cholinesterase inhibitor is memantine/donepezil. In some embodiments, the cholinesterase inhibitor is ambenonium. In some embodiments, the cholinesterase inhibitor is neostigmine.

In some embodiments, the dose of the therapeutic agents that treat or prevent cognitive impairment can be reduced by about 10%, by about 20%, by about 30%, by about 40%, by about 50%, by about 60%, by about 70%, by about 80%, or by about 90% for subjects that are heterozygous for an ST6GALNAC5 missense variant nucleic acid molecule encoding an ST6GALNAC5 predicted loss-of-function polypeptide (i.e., a less than the standard dosage amount) compared to subjects that are ST6GALNAC5 reference (who may receive a standard dosage amount). In some embodiments, the dose of the therapeutic agents that treat or prevent cognitive impairment can be reduced by about 10%, by about 20%, by about 30%, by about 40%, or by about 50%. In addition, the dose of therapeutic agents that treat or prevent cognitive impairment in subjects that are heterozygous for an ST6GALNAC5 missense variant nucleic acid molecule encoding an ST6GALNAC5 predicted loss-of-function polypeptide can be administered less frequently compared to subjects that are ST6GALNAC5 reference.

Administration of the therapeutic agents that treat or prevent cognitive impairment and/or ST6GALNAC5 inhibitors can be repeated, for example, after one day, two days, three days, five days, one week, two weeks, three weeks, one month, five weeks, six weeks, seven weeks, eight weeks, two months, or three months. The repeated administration can be at the same dose or at a different dose. The administration can be repeated once, twice, three times, four times, five times, six times, seven times, eight times, nine times, ten times, or more. For example, according to certain dosage regimens a subject can receive therapy for a prolonged period of time such as, for example, 6 months, 1 year, or more. In addition, the therapeutic agents that treat or prevent cognitive impairment and/or ST6GALNAC5 inhibitors can be administered sequentially or at the same time. In addition, the therapeutic agents that treat or prevent cognitive impairment and/or ST6GALNAC5 inhibitors can be administered in separate compositions or can be administered together in the same composition.

Administration of the therapeutic agents that treat or prevent cognitive impairment and/or ST6GALNAC5 inhibitors can occur by any suitable route including, but not limited to, parenteral, intravenous, oral, subcutaneous, intra-arterial, intracranial, intrathecal, intraperitoneal, topical, intranasal, or intramuscular. Pharmaceutical compositions for administration are desirably sterile and substantially isotonic and manufactured under GMP conditions. Pharmaceutical compositions can be provided in unit dosage form (i.e., the dosage for a single administration). Pharmaceutical compositions can be formulated using one or more physiologically and pharmaceutically acceptable carriers, diluents, excipients or auxiliaries. The formulation depends on the route of administration chosen. The term “pharmaceutically acceptable” means that the carrier, diluent, excipient, or auxiliary is compatible with the other ingredients of the formulation and not substantially deleterious to the recipient thereof.

The terms “treat”, “treating”, and “treatment” and “prevent”, “preventing”, and “prevention” as used herein, refer to eliciting the desired biological response, such as a therapeutic and prophylactic effect, respectively. In some embodiments, a therapeutic effect comprises one or more of a decrease/reduction in cognitive impairment, a decrease/reduction in the severity of cognitive impairment (such as, for example, a reduction or inhibition of development of cognitive impairment), a decrease/reduction in symptoms and cognitive impairment-related effects, delaying the onset of symptoms and cognitive impairment-related effects, reducing the severity of symptoms of cognitive impairment-related effects, reducing the severity of an acute episode, reducing the number of symptoms and cognitive impairment-related effects, reducing the latency of symptoms and cognitive impairment-related effects, an amelioration of symptoms and cognitive impairment-related effects, reducing secondary symptoms, reducing secondary infections, preventing relapse to cognitive impairment, decreasing the number or frequency of relapse episodes, increasing latency between symptomatic episodes, increasing time to sustained progression, expediting remission, inducing remission, augmenting remission, speeding recovery, or increasing efficacy of or decreasing resistance to alternative therapeutics, and/or an increased survival time of the affected host animal, following administration of the agent or composition comprising the agent. A prophylactic effect may comprise a complete or partial avoidance/inhibition or a delay of cognitive impairment development/progression (such as, for example, a complete or partial avoidance/inhibition or a delay), and an increased survival time of the affected host animal, following administration of a therapeutic protocol. Treatment of cognitive impairment encompasses the treatment of subjects already diagnosed as having any form of cognitive impairment at any clinical stage or manifestation, the delay of the onset or evolution or aggravation or deterioration of the symptoms or signs of cognitive impairment, and/or preventing and/or reducing the severity of cognitive impairment.

The present disclosure also provides methods of identifying a subject having an increased risk of developing cognitive impairment. In some embodiments, the methods comprise determining or having determined the presence or absence of an ST6GALNAC5 missense variant nucleic acid molecule (such as a genomic nucleic acid molecule, mRNA molecule, and/or cDNA molecule) encoding an ST6GALNAC5 predicted loss-of-function polypeptide in a biological sample obtained from the subject. When the subject lacks an ST6GALNAC5 missense variant nucleic acid molecule encoding an ST6GALNAC5 predicted loss-of-function polypeptide (i.e., the subject is genotypically categorized as ST6GALNAC5 reference), then the subject has an increased risk of developing cognitive impairment. When the subject has an ST6GALNAC5 missense variant nucleic acid molecule encoding an ST6GALNAC5 predicted loss-of-function polypeptide (i.e., the subject is heterozygous or homozygous for an ST6GALNAC5 missense variant nucleic acid molecule), then the subject has a decreased risk of developing cognitive impairment compared to a subject that is ST6GALNAC5 reference.

Having a single copy of an ST6GALNAC5 missense variant nucleic acid molecule encoding an ST6GALNAC5 predicted loss-of-function polypeptide is more protective of a subject from developing cognitive impairment than having no copies of an ST6GALNAC5 missense variant nucleic acid molecule encoding an ST6GALNAC5 predicted loss-of-function polypeptide. Without intending to be limited to any particular theory or mechanism of action, it is believed that a single copy of an ST6GALNAC5 missense variant nucleic acid molecule encoding an ST6GALNAC5 predicted loss-of-function polypeptide (i.e., heterozygous for an ST6GALNAC5 missense variant nucleic acid molecule) is protective of a subject from developing cognitive impairment, and it is also believed that having two copies of an ST6GALNAC5 missense variant nucleic acid molecule encoding an ST6GALNAC5 predicted loss-of-function polypeptide (i.e., homozygous for an ST6GALNAC5 missense variant nucleic acid molecule) may be more protective of a subject from developing cognitive impairment, relative to a subject with a single copy. Thus, in some embodiments, a single copy of an ST6GALNAC5 missense variant nucleic acid molecule encoding an ST6GALNAC5 predicted loss-of-function polypeptide may not be completely protective, but instead, may be partially or incompletely protective of a subject from developing cognitive impairment. While not desiring to be bound by any particular theory, there may be additional factors or molecules involved in the development of cognitive impairment that are still present in a subject having a single copy of an ST6GALNAC5 missense variant nucleic acid molecule encoding an ST6GALNAC5 predicted loss-of-function polypeptide, thus resulting in less than complete protection from the development of cognitive impairment.

Detecting the presence or absence of an ST6GALNAC5 missense variant nucleic acid molecule encoding an ST6GALNAC5 predicted loss-of-function polypeptide in a biological sample from the subject and/or determining whether a subject has an ST6GALNAC5 missense variant nucleic acid molecule encoding an ST6GALNAC5 predicted loss-of-function polypeptide can be carried out by any of the methods described herein. In some embodiments, these methods can be carried out in vitro. In some embodiments, these methods can be carried out in situ. In some embodiments, these methods can be carried out in vivo. In any of these embodiments, the ST6GALNAC5 missense variant nucleic acid molecule encoding an ST6GALNAC5 predicted loss-of-function polypeptide can be present within a cell obtained from the subject.

In some embodiments, when a subject is identified as having an increased risk of developing cognitive impairment, the subject is further treated with a therapeutic agent that treats or inhibits cognitive impairment and/or an ST6GALNAC5 inhibitor, as described herein. For example, when the subject is ST6GALNAC5 reference, and therefore has an increased risk of developing cognitive impairment, the subject is administered an ST6GALNAC5 inhibitor. In some embodiments, such a subject is administered a therapeutic agent that treats or inhibits cognitive impairment. In some embodiments, when the subject is heterozygous for an ST6GALNAC5 missense variant nucleic acid molecule encoding an ST6GALNAC5 predicted loss-of-function polypeptide, the subject is administered the therapeutic agent that treats or inhibits cognitive impairment in a dosage amount that is the same as or less than a standard dosage amount, and/or is administered an ST6GALNAC5 inhibitor. In some embodiments, the subject is ST6GALNAC5 reference. In some embodiments, the subject is heterozygous for an ST6GALNAC5 missense variant nucleic acid molecule encoding an ST6GALNAC5 predicted loss-of-function polypeptide.

The present disclosure also provides methods of detecting the presence or absence of an ST6GALNAC5 missense variant genomic nucleic acid molecule encoding an ST6GALNAC5 predicted loss-of-function polypeptide in a biological sample obtained from a subject, and/or an ST6GALNAC5 missense variant mRNA molecule encoding an ST6GALNAC5 predicted loss-of-function polypeptide in a biological sample obtained from a subject, and/or an ST6GALNAC5 missense variant cDNA molecule encoding an ST6GALNAC5 predicted loss-of-function polypeptide produced from an mRNA molecule in a biological sample obtained from a subject. It is understood that gene sequences within a population and mRNA molecules encoded by such genes can vary due to polymorphisms such as single-nucleotide polymorphisms. The sequences provided herein for the ST6GALNAC5 variant genomic nucleic acid molecule, ST6GALNAC5 variant mRNA molecule, and ST6GALNAC5 variant cDNA molecule are only exemplary sequences. Other sequences for the ST6GALNAC5 variant genomic nucleic acid molecule, variant mRNA molecule, and variant cDNA molecule are also possible.

The biological sample can be derived from any cell, tissue, or biological fluid from the subject. The biological sample may comprise any clinically relevant tissue such as, for example, a bone marrow sample, a tumor biopsy, a fine needle aspirate, or a sample of bodily fluid, such as blood, gingival crevicular fluid, plasma, serum, lymph, ascitic fluid, cystic fluid, or urine. In some embodiments, the biological sample comprises a buccal swab. The biological sample used in the methods disclosed herein can vary based on the assay format, nature of the detection method, and the tissues, cells, or extracts that are used as the sample. A biological sample can be processed differently depending on the assay being employed. For example, when detecting any ST6GALNAC5 variant nucleic acid molecule, preliminary processing designed to isolate or enrich the biological sample for the ST6GALNAC5 variant nucleic acid molecule can be employed. A variety of techniques may be used for this purpose. When detecting the level of any ST6GALNAC5 variant mRNA molecule, different techniques can be used enrich the biological sample with mRNA molecules. Various methods to detect the presence or level of an mRNA molecule or the presence of a particular variant genomic DNA locus can be used.

The present disclosure also provides methods of detecting an ST6GALNAC5 missense variant nucleic acid molecule, or the complement thereof, encoding an ST6GALNAC5 predicted loss-of-function polypeptide in a subject. The methods comprise assaying a biological sample obtained from the subject to determine whether a nucleic acid molecule in the biological sample is an ST6GALNAC5 missense variant nucleic acid molecule encoding an ST6GALNAC5 predicted loss-of-function polypeptide.

In some embodiments, the ST6GALNAC5 missense variant nucleic acid molecule encoding the ST6GALNAC5 predicted loss-of-function polypeptide, or the complement thereof, is a genomic nucleic acid molecule having a nucleotide sequence comprising a cytosine at a position corresponding to position 176,867 according to SEQ ID NO:2, or the complement thereof.

In some embodiments, the ST6GALNAC5 missense variant nucleic acid molecule encoding the ST6GALNAC5 predicted loss-of-function polypeptide, or the complement thereof, is an mRNA molecule having a nucleotide sequence comprising: a cytosine at a position corresponding to position 600 according to SEQ ID NO:11, or the complement thereof; a cytosine at a position corresponding to position 639 according to SEQ ID NO:12, or the complement thereof; a cytosine at a position corresponding to position 562 according to SEQ ID NO:13, or the complement thereof; a cytosine at a position corresponding to position 515 according to SEQ ID NO:14, or the complement thereof; a cytosine at a position corresponding to position 579 according to SEQ ID NO:15, or the complement thereof; a cytosine at a position corresponding to position 416 according to SEQ ID NO:16, or the complement thereof; a cytosine at a position corresponding to position 639 according to SEQ ID NO:17, or the complement thereof; or a cytosine at a position corresponding to position 600 according to SEQ ID NO:18, or the complement thereof.

In some embodiments, the ST6GALNAC5 missense variant nucleic acid molecule encoding the ST6GALNAC5 predicted loss-of-function polypeptide, or the complement thereof, is a cDNA molecule produced from an mRNA molecule in the biological sample having a nucleotide sequence comprising: a cytosine at a position corresponding to position 600 according to SEQ ID NO:27, or the complement thereof; a cytosine at a position corresponding to position 639 according to SEQ ID NO:28, or the complement thereof; a cytosine at a position corresponding to position 562 according to SEQ ID NO:29, or the complement thereof; a cytosine at a position corresponding to position 515 according to SEQ ID NO:30, or the complement thereof; a cytosine at a position corresponding to position 579 according to SEQ ID NO:31, or the complement thereof; a cytosine at a position corresponding to position 416 according to SEQ ID NO:32, or the complement thereof; a cytosine at a position corresponding to position 639 according to SEQ ID NO:33, or the complement thereof; or a cytosine at a position corresponding to position 600 according to SEQ ID NO:34, or the complement thereof.

In some embodiments, the ST6GALNAC5 missense variant nucleic acid molecule has a nucleotide sequence comprising: i) a cytosine at a position corresponding to position 176,867 according to SEQ ID NO:2 (for genomic nucleic acid molecules); ii) a cytosine at a position corresponding to position 600 according to SEQ ID NO:11; a cytosine at a position corresponding to position 639 according to SEQ ID NO:12; a cytosine at a position corresponding to position 562 according to SEQ ID NO:13; a cytosine at a position corresponding to position 515 according to SEQ ID NO:14; a cytosine at a position corresponding to position 579 according to SEQ ID NO:15; a cytosine at a position corresponding to position 416 according to SEQ ID NO:16; a cytosine at a position corresponding to position 639 according to SEQ ID NO:17; or a cytosine at a position corresponding to position 600 according to SEQ ID NO:18 (for mRNA molecules); or iii) a cytosine at a position corresponding to position 600 according to SEQ ID NO:27; a cytosine at a position corresponding to position 639 according to SEQ ID NO:28; a cytosine at a position corresponding to position 562 according to SEQ ID NO:29; a cytosine at a position corresponding to position 515 according to SEQ ID NO:30; a cytosine at a position corresponding to position 579 according to SEQ ID NO:31; a cytosine at a position corresponding to position 416 according to SEQ ID NO:32; a cytosine at a position corresponding to position 639 according to SEQ ID NO:33; or a cytosine at a position corresponding to position 600 according to SEQ ID NO:34 (for cDNA molecules obtained from mRNA molecules).

In some embodiments, the biological sample comprises a cell or cell lysate. Such methods can further comprise, for example, obtaining a biological sample from the subject comprising an ST6GALNAC5 genomic nucleic acid molecule or mRNA molecule, and if mRNA, optionally reverse transcribing the mRNA into cDNA. Such assays can comprise, for example determining the identity of these positions of the particular ST6GALNAC5 nucleic acid molecule. In some embodiments, the method is an in vitro method.

In some embodiments, the determining step, detecting step, or sequence analysis comprises sequencing at least a portion of the nucleotide sequence of the ST6GALNAC5 genomic nucleic acid molecule, the ST6GALNAC5 mRNA molecule, or the ST6GALNAC5 cDNA molecule produced from the mRNA molecule in the biological sample, wherein the sequenced portion comprises one or more variations that cause a loss-of-function (partial or complete) or are predicted to cause a loss-of-function (partial or complete).

In some embodiments, the determining step, detecting step, or sequence analysis comprises sequencing at least a portion of: i) the nucleotide sequence of the ST6GALNAC5 genomic nucleic acid molecule in the biological sample, wherein the sequenced portion comprises a position corresponding to position 176,867 according to SEQ ID NO:2, or the complement thereof; ii) the nucleotide sequence of the ST6GALNAC5 mRNA molecule in the biological sample, wherein the sequenced portion comprises a position corresponding to: position 600 according to SEQ ID NO:11, or the complement thereof; position 639 according to SEQ ID NO:12, or the complement thereof; position 562 according to SEQ ID NO:13, or the complement thereof; position 515 according to SEQ ID NO:14, or the complement thereof; position 579 according to SEQ ID NO:15, or the complement thereof; position 416 according to SEQ ID NO:16, or the complement thereof; position 639 according to SEQ ID NO:17, or the complement thereof; or position 600 according to SEQ ID NO:18, or the complement thereof; and/or iii) the nucleotide sequence of the ST6GALNAC5 cDNA molecule produced from the mRNA in the biological sample, wherein the sequenced portion comprises a position corresponding to: position 600 according to SEQ ID NO:27, or the complement thereof; position 639 according to SEQ ID NO:28, or the complement thereof; position 562 according to SEQ ID NO:29, or the complement thereof; position 515 according to SEQ ID NO:30, or the complement thereof; position 579 according to SEQ ID NO:31, or the complement thereof; position 416 according to SEQ ID NO:32, or the complement thereof; position 639 according to SEQ ID NO:33, or the complement thereof; or position 600 according to SEQ ID NO:34, or the complement thereof. When the sequenced portion of the ST6GALNAC5 nucleic acid molecule in the biological sample comprises: a cytosine at a position corresponding to position 176,867 according to SEQ ID NO:2, a cytosine at a position corresponding to position 600 according to SEQ ID NO:11, a cytosine at a position corresponding to position 639 according to SEQ ID NO:12, a cytosine at a position corresponding to position 562 according to SEQ ID NO:13, a cytosine at a position corresponding to position 515 according to SEQ ID NO:14, a cytosine at a position corresponding to position 579 according to SEQ ID NO:15, a cytosine at a position corresponding to position 416 according to SEQ ID NO:16, a cytosine at a position corresponding to position 639 according to SEQ ID NO:17, a cytosine at a position corresponding to position 600 according to SEQ ID NO:18, a cytosine at a position corresponding to position 600 according to SEQ ID NO:27, a cytosine at a position corresponding to position 639 according to SEQ ID NO:28, a cytosine at a position corresponding to position 562 according to SEQ ID NO:29, a cytosine at a position corresponding to position 515 according to SEQ ID NO:30, a cytosine at a position corresponding to position 579 according to SEQ ID NO:31, a cytosine at a position corresponding to position 416 according to SEQ ID NO:32, a cytosine at a position corresponding to position 639 according to SEQ ID NO:33, or a cytosine at a position corresponding to position 600 according to SEQ ID NO:34, then the ST6GALNAC5 nucleic acid molecule in the biological sample is an ST6GALNAC5 missense variant nucleic acid molecule encoding an ST6GALNAC5 predicted loss-of-function polypeptide.

In some embodiments, the determining step, detecting step, or sequence analysis comprises sequencing at least a portion of the nucleotide sequence of the ST6GALNAC5 genomic nucleic acid molecule in the biological sample, wherein the sequenced portion comprises a position corresponding to position 176,867 according to SEQ ID NO:2, or the complement thereof. When the sequenced portion of the ST6GALNAC5 nucleic acid molecule in the biological sample comprises a cytosine at a position corresponding to position 176,867 according to SEQ ID NO:2, then the ST6GALNAC5 nucleic acid molecule in the biological sample is an ST6GALNAC5 missense variant genomic nucleic acid molecule encoding an ST6GALNAC5 predicted loss-of-function polypeptide.

In some embodiments, the determining step, detecting step, or sequence analysis comprises sequencing at least a portion of the nucleotide sequence of the ST6GALNAC5 mRNA molecule in the biological sample, wherein the sequenced portion comprises a position corresponding to: position 600 according to SEQ ID NO:11, or the complement thereof; position 639 according to SEQ ID NO:12, or the complement thereof; position 562 according to SEQ ID NO:13, or the complement thereof; position 515 according to SEQ ID NO:14, or the complement thereof; position 579 according to SEQ ID NO:15, or the complement thereof; position 416 according to SEQ ID NO:16, or the complement thereof; position 639 according to SEQ ID NO:17, or the complement thereof; or position 600 according to SEQ ID NO:18, or the complement thereof. When the sequenced portion of the ST6GALNAC5 mRNA molecule in the biological sample comprises: a cytosine at a position corresponding to position 600 according to SEQ ID NO:11, a cytosine at a position corresponding to position 639 according to SEQ ID NO:12, a cytosine at a position corresponding to position 562 according to SEQ ID NO:13, a cytosine at a position corresponding to position 515 according to SEQ ID NO:14, a cytosine at a position corresponding to position 579 according to SEQ ID NO:15, a cytosine at a position corresponding to position 416 according to SEQ ID NO:16, a cytosine at a position corresponding to position 639 according to SEQ ID NO:17, or a cytosine at a position corresponding to position 600 according to SEQ ID NO:18, then the ST6GALNAC5 nucleic acid molecule in the biological sample is an ST6GALNAC5 missense variant mRNA molecule encoding an ST6GALNAC5 predicted loss-of-function polypeptide.

In some embodiments, the determining step, detecting step, or sequence analysis comprises sequencing at least a portion of the nucleotide sequence of the ST6GALNAC5 cDNA molecule produced from the mRNA molecule in the biological sample, wherein the sequenced portion comprises a position corresponding to: position 600 according to SEQ ID NO:27, or the complement thereof; position 639 according to SEQ ID NO:28, or the complement thereof; position 562 according to SEQ ID NO:29, or the complement thereof; position 515 according to SEQ ID NO:30, or the complement thereof; position 579 according to SEQ ID NO:31, or the complement thereof; position 416 according to SEQ ID NO:32, or the complement thereof; position 639 according to SEQ ID NO:33, or the complement thereof; or position 600 according to SEQ ID NO:34, or the complement thereof. When the sequenced portion of the ST6GALNAC5 cDNA molecule in the biological sample comprises: a cytosine at a position corresponding to position 600 according to SEQ ID NO:27, a cytosine at a position corresponding to position 639 according to SEQ ID NO:28, a cytosine at a position corresponding to position 562 according to SEQ ID NO:29, a cytosine at a position corresponding to position 515 according to SEQ ID NO:30, a cytosine at a position corresponding to position 579 according to SEQ ID NO:31, a cytosine at a position corresponding to position 416 according to SEQ ID NO:32, a cytosine at a position corresponding to position 639 according to SEQ ID NO:33, or a cytosine at a position corresponding to position 600 according to SEQ ID NO:34, then the ST6GALNAC5 nucleic acid molecule in the biological sample is an ST6GALNAC5 missense variant cDNA molecule encoding an ST6GALNAC5 predicted loss-of-function polypeptide.

In some embodiments, the determining step, detecting step, or sequence analysis comprises: a) contacting the biological sample with a primer hybridizing to a portion of the nucleotide sequence of the ST6GALNAC5: i) genomic nucleic acid molecule, or the complement thereof, that is proximate to a position corresponding to position 176,867 according to SEQ ID NO:2, or the complement thereof; ii) mRNA molecule, or the complement thereof, that is proximate to a position corresponding to: position 600 according to SEQ ID NO:11, or the complement thereof; position 639 according to SEQ ID NO:12, or the complement thereof; position 562 according to SEQ ID NO:13, or the complement thereof; position 515 according to SEQ ID NO:14, or the complement thereof; position 579 according to SEQ ID NO:15, or the complement thereof; position 416 according to SEQ ID NO:16, or the complement thereof; position 639 according to SEQ ID NO:17, or the complement thereof; or position 600 according to SEQ ID NO:18, or the complement thereof; and/or iii) cDNA molecule, or the complement thereof, that is proximate to a position corresponding to: position 600 according to SEQ ID NO:27, or the complement thereof; position 639 according to SEQ ID NO:28, or the complement thereof; position 562 according to SEQ ID NO:29, or the complement thereof; position 515 according to SEQ ID NO:30, or the complement thereof; position 579 according to SEQ ID NO:31, or the complement thereof; position 416 according to SEQ ID NO:32, or the complement thereof; position 639 according to SEQ ID NO:33, or the complement thereof; or position 600 according to SEQ ID NO:34, or the complement thereof; b) extending the primer at least through the position of the nucleotide sequence of the ST6GALNAC5: i) genomic nucleic acid molecule, or the complement thereof, corresponding to position 176,867 according to SEQ ID NO:2, or the complement thereof; ii) mRNA molecule, or the complement thereof, corresponding to: position 600 according to SEQ ID NO:11, or the complement thereof; position 639 according to SEQ ID NO:12, or the complement thereof; position 562 according to SEQ ID NO:13, or the complement thereof; position 515 according to SEQ ID NO:14, or the complement thereof; position 579 according to SEQ ID NO:15, or the complement thereof; position 416 according to SEQ ID NO:16, or the complement thereof; position 639 according to SEQ ID NO:17, or the complement thereof; or position 600 according to SEQ ID NO:18, or the complement thereof; and/or iii) cDNA molecule, or the complement thereof, corresponding to: position 600 according to SEQ ID NO:27, or the complement thereof; position 639 according to SEQ ID NO:28, or the complement thereof; position 562 according to SEQ ID NO:29, or the complement thereof; position 515 according to SEQ ID NO:30, or the complement thereof; position 579 according to SEQ ID NO:31, or the complement thereof; position 416 according to SEQ ID NO:32, or the complement thereof; position 639 according to SEQ ID NO:33, or the complement thereof; or position 600 according to SEQ ID NO:34, or the complement thereof; and c) determining whether the extension product of the primer comprises: a cytosine at a position corresponding to position 176,867 according to SEQ ID NO:2, or the complement thereof; a cytosine at a position corresponding to position 600 according to SEQ ID NO:11, or the complement thereof; a cytosine at a position corresponding to position 639 according to SEQ ID NO:12, or the complement thereof; a cytosine at a position corresponding to position 562 according to SEQ ID NO:13, or the complement thereof; a cytosine at a position corresponding to position 515 according to SEQ ID NO:14, or the complement thereof; a cytosine at a position corresponding to position 579 according to SEQ ID NO:15, or the complement thereof; a cytosine at a position corresponding to position 416 according to SEQ ID NO:16, or the complement thereof; a cytosine at a position corresponding to position 639 according to SEQ ID NO:17, or the complement thereof; a cytosine at a position corresponding to position 600 according to SEQ ID NO:18, or the complement thereof; a cytosine at a position corresponding to position 600 according to SEQ ID NO:27, or the complement thereof; a cytosine at a position corresponding to position 639 according to SEQ ID NO:28, or the complement thereof; a cytosine at a position corresponding to position 562 according to SEQ ID NO:29, or the complement thereof; a cytosine at a position corresponding to position 515 according to SEQ ID NO:30, or the complement thereof; a cytosine at a position corresponding to position 579 according to SEQ ID NO:31, or the complement thereof; a cytosine at a position corresponding to position 416 according to SEQ ID NO:32, or the complement thereof; a cytosine at a position corresponding to position 639 according to SEQ ID NO:33, or the complement thereof; or a cytosine at a position corresponding to position 600 according to SEQ ID NO:34, or the complement thereof.

In some embodiments, the determining step, detecting step, or sequence analysis comprises: a) contacting the biological sample with a primer hybridizing to a portion of the nucleotide sequence of the ST6GALNAC5 genomic nucleic acid molecule, or the complement thereof, that is proximate to a position corresponding to position 176,867 according to SEQ ID NO:2, or the complement thereof; b) extending the primer at least through the position of the nucleotide sequence of the ST6GALNAC5 genomic nucleic acid molecule, or the complement thereof, corresponding to position 176,867 according to SEQ ID NO:2, or the complement thereof; and c) determining whether the extension product of the primer comprises a cytosine at a position corresponding to position 176,867 according to SEQ ID NO:2, or the complement thereof.

In some embodiments, the determining step, detecting step, or sequence analysis comprises: a) contacting the biological sample with a primer hybridizing to a portion of the nucleotide sequence of the ST6GALNAC5 mRNA molecule, or the complement thereof, that is proximate to a position corresponding to: position 600 according to SEQ ID NO:11, or the complement thereof; position 639 according to SEQ ID NO:12, or the complement thereof; position 562 according to SEQ ID NO:13, or the complement thereof; position 515 according to SEQ ID NO:14, or the complement thereof; position 579 according to SEQ ID NO:15, or the complement thereof; position 416 according to SEQ ID NO:16, or the complement thereof; position 639 according to SEQ ID NO:17, or the complement thereof; or position 600 according to SEQ ID NO:18, or the complement thereof; b) extending the primer at least through the position of the nucleotide sequence of the ST6GALNAC5 mRNA molecule corresponding to: position 600 according to SEQ ID NO:11, or the complement thereof; position 639 according to SEQ ID NO:12, or the complement thereof; position 562 according to SEQ ID NO:13, or the complement thereof; position 515 according to SEQ ID NO:14, or the complement thereof; position 579 according to SEQ ID NO:15, or the complement thereof; position 416 according to SEQ ID NO:16, or the complement thereof; position 639 according to SEQ ID NO:17, or the complement thereof; or position 600 according to SEQ ID NO:18, or the complement thereof; and c) determining whether the extension product of the primer comprises: a cytosine at a position corresponding to position 600 according to SEQ ID NO:11, or the complement thereof; a cytosine at a position corresponding to position 639 according to SEQ ID NO:12, or the complement thereof; a cytosine at a position corresponding to position 562 according to SEQ ID NO:13, or the complement thereof; a cytosine at a position corresponding to position 515 according to SEQ ID NO:14, or the complement thereof; a cytosine at a position corresponding to position 579 according to SEQ ID NO:15, or the complement thereof; a cytosine at a position corresponding to position 416 according to SEQ ID NO:16, or the complement thereof; a cytosine at a position corresponding to position 639 according to SEQ ID NO:17, or the complement thereof; or a cytosine at a position corresponding to position 600 according to SEQ ID NO:18, or the complement thereof.

In some embodiments, the determining step, detecting step, or sequence analysis comprises: a) contacting the biological sample with a primer hybridizing to a portion of the nucleotide sequence of the ST6GALNAC5 cDNA molecule, or the complement thereof, that is proximate to a position corresponding to: position 600 according to SEQ ID NO:27, or the complement thereof; position 639 according to SEQ ID NO:28, or the complement thereof; position 562 according to SEQ ID NO:29, or the complement thereof; position 515 according to SEQ ID NO:30, or the complement thereof; position 579 according to SEQ ID NO:31, or the complement thereof; position 416 according to SEQ ID NO:32, or the complement thereof; position 639 according to SEQ ID NO:33, or the complement thereof; or position 600 according to SEQ ID NO:34, or the complement thereof; b) extending the primer at least through the position of the nucleotide sequence of the ST6GALNAC5 cDNA molecule corresponding to: position 600 according to SEQ ID NO:27, or the complement thereof; position 639 according to SEQ ID NO:28, or the complement thereof; position 562 according to SEQ ID NO:29, or the complement thereof; position 515 according to SEQ ID NO:30, or the complement thereof; position 579 according to SEQ ID NO:31, or the complement thereof; position 416 according to SEQ ID NO:32, or the complement thereof; position 639 according to SEQ ID NO:33, or the complement thereof; or position 600 according to SEQ ID NO:34, or the complement thereof; and c) determining whether the extension product of the primer comprises: a cytosine at a position corresponding to position 600 according to SEQ ID NO:27, or the complement thereof; a cytosine at a position corresponding to position 639 according to SEQ ID NO:28, or the complement thereof; a cytosine at a position corresponding to position 562 according to SEQ ID NO:29, or the complement thereof; a cytosine at a position corresponding to position 515 according to SEQ ID NO:30, or the complement thereof; a cytosine at a position corresponding to position 579 according to SEQ ID NO:31, or the complement thereof; a cytosine at a position corresponding to position 416 according to SEQ ID NO:32, or the complement thereof; a cytosine at a position corresponding to position 639 according to SEQ ID NO:33, or the complement thereof; or a cytosine at a position corresponding to position 600 according to SEQ ID NO:34, or the complement thereof.

In some embodiments, the entire nucleic acid molecule is sequenced. In some embodiments, only an ST6GALNAC5 genomic nucleic acid molecule is analyzed. In some embodiments, only an ST6GALNAC5 mRNA is analyzed. In some embodiments, only an ST6GALNAC5 cDNA obtained from ST6GALNAC5 mRNA is analyzed.

In some embodiments, the determining step, detecting step, or sequence analysis comprises: a) amplifying at least a portion of the ST6GALNAC5 nucleic acid molecule, or the complement thereof, in the biological sample, wherein the amplified portion comprises: a cytosine at a position corresponding to position 176,867 according to SEQ ID NO:2, or the complement thereof; a cytosine at a position corresponding to position 600 according to SEQ ID NO:11, or the complement thereof; a cytosine at a position corresponding to position 639 according to SEQ ID NO:12, or the complement thereof; a cytosine at a position corresponding to position 562 according to SEQ ID NO:13, or the complement thereof; a cytosine at a position corresponding to position 515 according to SEQ ID NO:14, or the complement thereof; a cytosine at a position corresponding to position 579 according to SEQ ID NO:15, or the complement thereof; a cytosine at a position corresponding to position 416 according to SEQ ID NO:16, or the complement thereof; a cytosine at a position corresponding to position 639 according to SEQ ID NO:17, or the complement thereof; a cytosine at a position corresponding to position 600 according to SEQ ID NO:18, or the complement thereof; a cytosine at a position corresponding to position 600 according to SEQ ID NO:27, or the complement thereof; a cytosine at a position corresponding to position 639 according to SEQ ID NO:28, or the complement thereof; a cytosine at a position corresponding to position 562 according to SEQ ID NO:29, or the complement thereof; a cytosine at a position corresponding to position 515 according to SEQ ID NO:30, or the complement thereof; a cytosine at a position corresponding to position 579 according to SEQ ID NO:31, or the complement thereof; a cytosine at a position corresponding to position 416 according to SEQ ID NO:32, or the complement thereof; a cytosine at a position corresponding to position 639 according to SEQ ID NO:33, or the complement thereof; or a cytosine at a position corresponding to position 600 according to SEQ ID NO:34, or the complement thereof; b) labeling the amplified nucleic acid molecule with a detectable label; c) contacting the labeled nucleic acid molecule with a support comprising an alteration-specific probe, wherein the alteration-specific probe comprises a nucleotide sequence which hybridizes under stringent conditions to the nucleic acid sequence of the amplified nucleic acid molecule comprising: a cytosine at a position corresponding to position 176,867 according to SEQ ID NO:2, or the complement thereof; a cytosine at a position corresponding to position 600 according to SEQ ID NO:11, or the complement thereof; a cytosine at a position corresponding to position 639 according to SEQ ID NO:12, or the complement thereof; a cytosine at a position corresponding to position 562 according to SEQ ID NO:13, or the complement thereof; a cytosine at a position corresponding to position 515 according to SEQ ID NO:14, or the complement thereof; a cytosine at a position corresponding to position 579 according to SEQ ID NO:15, or the complement thereof; a cytosine at a position corresponding to position 416 according to SEQ ID NO:16, or the complement thereof; a cytosine at a position corresponding to position 639 according to SEQ ID NO:17, or the complement thereof; a cytosine at a position corresponding to position 600 according to SEQ ID NO:18, or the complement thereof; a cytosine at a position corresponding to position 600 according to SEQ ID NO:27, or the complement thereof; a cytosine at a position corresponding to position 639 according to SEQ ID NO:28, or the complement thereof; a cytosine at a position corresponding to position 562 according to SEQ ID NO:29, or the complement thereof; a cytosine at a position corresponding to position 515 according to SEQ ID NO:30, or the complement thereof; a cytosine at a position corresponding to position 579 according to SEQ ID NO:31, or the complement thereof; a cytosine at a position corresponding to position 416 according to SEQ ID NO:32, or the complement thereof; a cytosine at a position corresponding to position 639 according to SEQ ID NO:33, or the complement thereof; or a cytosine at a position corresponding to position 600 according to SEQ ID NO:34, or the complement thereof; and d) detecting the detectable label.

In some embodiments, the determining step, detecting step, or sequence analysis comprises: a) amplifying at least a portion of the ST6GALNAC5 genomic nucleic acid molecule, or the complement thereof, in the biological sample, wherein the portion comprises a cytosine at a position corresponding to position 176,867 according to SEQ ID NO:2, or the complement thereof; b) labeling the amplified nucleic acid molecule with a detectable label; c) contacting the labeled nucleic acid molecule with a support comprising an alteration-specific probe, wherein the alteration-specific probe comprises a nucleotide sequence which hybridizes under stringent conditions to the nucleic acid sequence of the amplified nucleic acid molecule comprising a cytosine at a position corresponding to position 176,867 according to SEQ ID NO:2, or the complement thereof; and d) detecting the detectable label.

In some embodiments, the determining step, detecting step, or sequence analysis comprises: a) amplifying at least a portion of the ST6GALNAC5 mRNA molecule, or the complement thereof, in the biological sample, wherein the portion comprises: a cytosine at a position corresponding to position 600 according to SEQ ID NO:11, or the complement thereof; a cytosine at a position corresponding to position 639 according to SEQ ID NO:12, or the complement thereof; a cytosine at a position corresponding to position 562 according to SEQ ID NO:13, or the complement thereof; a cytosine at a position corresponding to position 515 according to SEQ ID NO:14, or the complement thereof; a cytosine at a position corresponding to position 579 according to SEQ ID NO:15, or the complement thereof; a cytosine at a position corresponding to position 416 according to SEQ ID NO:16, or the complement thereof; a cytosine at a position corresponding to position 639 according to SEQ ID NO:17, or the complement thereof; or a cytosine at a position corresponding to position 600 according to SEQ ID NO:18, or the complement thereof; b) labeling the amplified nucleic acid molecule with a detectable label; c) contacting the labeled nucleic acid molecule with a support comprising an alteration-specific probe, wherein the alteration-specific probe comprises a nucleotide sequence which hybridizes under stringent conditions to the nucleic acid sequence of the amplified nucleic acid molecule comprising: a cytosine at a position corresponding to position 600 according to SEQ ID NO:11, or the complement thereof; a cytosine at a position corresponding to position 639 according to SEQ ID NO:12, or the complement thereof; a cytosine at a position corresponding to position 562 according to SEQ ID NO:13, or the complement thereof; a cytosine at a position corresponding to position 515 according to SEQ ID NO:14, or the complement thereof; a cytosine at a position corresponding to position 579 according to SEQ ID NO:15, or the complement thereof; a cytosine at a position corresponding to position 416 according to SEQ ID NO:16, or the complement thereof; a cytosine at a position corresponding to position 639 according to SEQ ID NO:17, or the complement thereof; or a cytosine at a position corresponding to position 600 according to SEQ ID NO:18, or the complement thereof; and d) detecting the detectable label.

In some embodiments, the determining step, detecting step, or sequence analysis comprises: a) amplifying at least a portion of the ST6GALNAC5 cDNA molecule, or the complement thereof, in the biological sample, wherein the portion comprises: a cytosine at a position corresponding to position 600 according to SEQ ID NO:27, or the complement thereof; a cytosine at a position corresponding to position 639 according to SEQ ID NO:28, or the complement thereof; a cytosine at a position corresponding to position 562 according to SEQ ID NO:29, or the complement thereof; a cytosine at a position corresponding to position 515 according to SEQ ID NO:30, or the complement thereof; a cytosine at a position corresponding to position 579 according to SEQ ID NO:31, or the complement thereof; a cytosine at a position corresponding to position 416 according to SEQ ID NO:32, or the complement thereof; a cytosine at a position corresponding to position 639 according to SEQ ID NO:33, or the complement thereof; or a cytosine at a position corresponding to position 600 according to SEQ ID NO:34, or the complement thereof; b) labeling the amplified nucleic acid molecule with a detectable label; c) contacting the labeled nucleic acid molecule with a support comprising an alteration-specific probe, wherein the alteration-specific probe comprises a nucleotide sequence which hybridizes under stringent conditions to the nucleic acid sequence of the amplified nucleic acid molecule comprising: a cytosine at a position corresponding to position 600 according to SEQ ID NO:27, or the complement thereof; a cytosine at a position corresponding to position 639 according to SEQ ID NO:28, or the complement thereof; a cytosine at a position corresponding to position 562 according to SEQ ID NO:29, or the complement thereof; a cytosine at a position corresponding to position 515 according to SEQ ID NO:30, or the complement thereof; a cytosine at a position corresponding to position 579 according to SEQ ID NO:31, or the complement thereof; a cytosine at a position corresponding to position 416 according to SEQ ID NO:32, or the complement thereof; a cytosine at a position corresponding to position 639 according to SEQ ID NO:33, or the complement thereof; or a cytosine at a position corresponding to position 600 according to SEQ ID NO:34, or the complement thereof; and d) detecting the detectable label.

In some embodiments, the nucleic acid molecule is mRNA and the determining step further comprises reverse-transcribing the mRNA into a cDNA prior to the amplifying step.

In some embodiments, the determining step, detecting step, or sequence analysis comprises: contacting the ST6GALNAC5 nucleic acid molecule, or the complement thereof, in the biological sample with an alteration-specific probe comprising a detectable label, wherein the alteration-specific probe comprises a nucleotide sequence which hybridizes under stringent conditions to the nucleotide sequence of the ST6GALNAC5 nucleic acid molecule, or the complement thereof, comprising: a cytosine at a position corresponding to position 176,867 according to SEQ ID NO:2, or the complement thereof; a cytosine at a position corresponding to position 600 according to SEQ ID NO:11, or the complement thereof; a cytosine at a position corresponding to position 639 according to SEQ ID NO:12, or the complement thereof; a cytosine at a position corresponding to position 562 according to SEQ ID NO:13, or the complement thereof; a cytosine at a position corresponding to position 515 according to SEQ ID NO:14, or the complement thereof; a cytosine at a position corresponding to position 579 according to SEQ ID NO:15, or the complement thereof; a cytosine at a position corresponding to position 416 according to SEQ ID NO:16, or the complement thereof; a cytosine at a position corresponding to position 639 according to SEQ ID NO:17, or the complement thereof; a cytosine at a position corresponding to position 600 according to SEQ ID NO:18, or the complement thereof; a cytosine at a position corresponding to position 600 according to SEQ ID NO:27, or the complement thereof; a cytosine at a position corresponding to position 639 according to SEQ ID NO:28, or the complement thereof; a cytosine at a position corresponding to position 562 according to SEQ ID NO:29, or the complement thereof; a cytosine at a position corresponding to position 515 according to SEQ ID NO:30, or the complement thereof; a cytosine at a position corresponding to position 579 according to SEQ ID NO:31, or the complement thereof; a cytosine at a position corresponding to position 416 according to SEQ ID NO:32, or the complement thereof; a cytosine at a position corresponding to position 639 according to SEQ ID NO:33, or the complement thereof; or a cytosine at a position corresponding to position 600 according to SEQ ID NO:34, or the complement thereof; and detecting the detectable label.

In some embodiments, the determining step, detecting step, or sequence analysis comprises: contacting the ST6GALNAC5 genomic nucleic acid molecule, or the complement thereof, in the biological sample with an alteration-specific probe comprising a detectable label, wherein the alteration-specific probe comprises a nucleotide sequence which hybridizes under stringent conditions to the nucleotide sequence of the ST6GALNAC5 genomic nucleic acid molecule, or the complement thereof, comprising a cytosine at a position corresponding to position 176,867 according to SEQ ID NO:2, or the complement thereof; and detecting the detectable label.

In some embodiments, the determining step, detecting step, or sequence analysis comprises: contacting the ST6GALNAC5 mRNA molecule, or the complement thereof, in the biological sample with an alteration-specific probe comprising a detectable label, wherein the alteration-specific probe comprises a nucleotide sequence which hybridizes under stringent conditions to the nucleotide sequence of the ST6GALNAC5 mRNA molecule, or the complement thereof, comprising: a cytosine at a position corresponding to position 600 according to SEQ ID NO:11, or the complement thereof; a cytosine at a position corresponding to position 639 according to SEQ ID NO:12, or the complement thereof; a cytosine at a position corresponding to position 562 according to SEQ ID NO:13, or the complement thereof; a cytosine at a position corresponding to position 515 according to SEQ ID NO:14, or the complement thereof; a cytosine at a position corresponding to position 579 according to SEQ ID NO:15, or the complement thereof; a cytosine at a position corresponding to position 416 according to SEQ ID NO:16, or the complement thereof; a cytosine at a position corresponding to position 639 according to SEQ ID NO:17, or the complement thereof; or a cytosine at a position corresponding to position 600 according to SEQ ID NO:18, or the complement thereof; and detecting the detectable label.

In some embodiments, the determining step, detecting step, or sequence analysis comprises: contacting the ST6GALNAC5 cDNA molecule, or the complement thereof, produced from an mRNA molecule in the biological sample with an alteration-specific probe comprising a detectable label, wherein the alteration-specific probe comprises a nucleotide sequence which hybridizes under stringent conditions to the nucleotide sequence of the ST6GALNAC5 cDNA molecule, or the complement thereof, comprising: a cytosine at a position corresponding to position 600 according to SEQ ID NO:27, or the complement thereof; a cytosine at a position corresponding to position 639 according to SEQ ID NO:28, or the complement thereof; a cytosine at a position corresponding to position 562 according to SEQ ID NO:29, or the complement thereof; a cytosine at a position corresponding to position 515 according to SEQ ID NO:30, or the complement thereof; a cytosine at a position corresponding to position 579 according to SEQ ID NO:31, or the complement thereof; a cytosine at a position corresponding to position 416 according to SEQ ID NO:32, or the complement thereof; a cytosine at a position corresponding to position 639 according to SEQ ID NO:33, or the complement thereof; or a cytosine at a position corresponding to position 600 according to SEQ ID NO:34, or the complement thereof; and detecting the detectable label.

In some embodiments, the ST6GALNAC5 nucleic acid molecule is present within a cell obtained from the subject.

Alteration-specific polymerase chain reaction techniques can be used to detect mutations such as SNPs in a nucleic acid sequence. Alteration-specific primers can be used because the DNA polymerase will not extend when a mismatch with the template is present.

In some embodiments, the determining step, detecting step, or sequence analysis comprises contacting the biological sample with a primer or probe, such as an alteration-specific primer or alteration-specific probe, that specifically hybridizes to an ST6GALNAC5 variant genomic sequence, variant mRNA sequence, or variant cDNA sequence and not the corresponding ST6GALNAC5 reference sequence under stringent conditions, and determining whether hybridization has occurred.

In some embodiments, the assay comprises RNA sequencing (RNA-Seq). In some embodiments, the assays also comprise reverse transcribing mRNA into cDNA, such as by the reverse transcriptase polymerase chain reaction (RT-PCR).

In some embodiments, the methods utilize probes and primers of sufficient nucleotide length to bind to the target nucleotide sequence and specifically detect and/or identify a polynucleotide comprising an ST6GALNAC5 variant genomic nucleic acid molecule, variant mRNA molecule, or variant cDNA molecule. The hybridization conditions or reaction conditions can be determined by the operator to achieve this result. The nucleotide length may be any length that is sufficient for use in a detection method of choice, including any assay described or exemplified herein. Such probes and primers can hybridize specifically to a target nucleotide sequence under high stringency hybridization conditions. Probes and primers may have complete nucleotide sequence identity of contiguous nucleotides within the target nucleotide sequence, although probes differing from the target nucleotide sequence and that retain the ability to specifically detect and/or identify a target nucleotide sequence may be designed by conventional methods. Probes and primers can have about 80%, about 85%, about 90%, about 91%, about 92%, about 93%, about 94%, about 95%, about 96%, about 97%, about 98%, about 99%, or 100% sequence identity or complementarity with the nucleotide sequence of the target nucleic acid molecule.

In some embodiments, to determine whether an ST6GALNAC5 nucleic acid molecule (genomic nucleic acid molecule, mRNA molecule, or cDNA molecule), or complement thereof, within a biological sample comprises a nucleotide sequence comprising a cytosine at a position corresponding to position 176,867 according to SEQ ID NO:2, a cytosine at a position corresponding to position 600 according to SEQ ID NO:11, a cytosine at a position corresponding to position 639 according to SEQ ID NO:12, a cytosine at a position corresponding to position 562 according to SEQ ID NO:13, a cytosine at a position corresponding to position 515 according to SEQ ID NO:14, a cytosine at a position corresponding to position 579 according to SEQ ID NO:15, a cytosine at a position corresponding to position 416 according to SEQ ID NO:16, a cytosine at a position corresponding to position 639 according to SEQ ID NO:17, a cytosine at a position corresponding to position 600 according to SEQ ID NO:18, a cytosine at a position corresponding to position 600 according to SEQ ID NO:27, a cytosine at a position corresponding to position 639 according to SEQ ID NO:28, a cytosine at a position corresponding to position 562 according to SEQ ID NO:29, a cytosine at a position corresponding to position 515 according to SEQ ID NO:30, a cytosine at a position corresponding to position 579 according to SEQ ID NO:31, a cytosine at a position corresponding to position 416 according to SEQ ID NO:32, a cytosine at a position corresponding to position 639 according to SEQ ID NO:33, or a cytosine at a position corresponding to position 600 according to SEQ ID NO:34, the biological sample can be subjected to an amplification method using a primer pair that includes a first primer derived from the 5′ flanking sequence adjacent to a cytosine at a position corresponding to position 176,867 according to SEQ ID NO:2, a cytosine at a position corresponding to position 600 according to SEQ ID NO:11, a cytosine at a position corresponding to position 639 according to SEQ ID NO:12, a cytosine at a position corresponding to position 562 according to SEQ ID NO:13, a cytosine at a position corresponding to position 515 according to SEQ ID NO:14, a cytosine at a position corresponding to position 579 according to SEQ ID NO:15, a cytosine at a position corresponding to position 416 according to SEQ ID NO:16, a cytosine at a position corresponding to position 639 according to SEQ ID NO:17, a cytosine at a position corresponding to position 600 according to SEQ ID NO:18, a cytosine at a position corresponding to position 600 according to SEQ ID NO:27, a cytosine at a position corresponding to position 639 according to SEQ ID NO:28, a cytosine at a position corresponding to position 562 according to SEQ ID NO:29, a cytosine at a position corresponding to position 515 according to SEQ ID NO:30, a cytosine at a position corresponding to position 579 according to SEQ ID NO:31, a cytosine at a position corresponding to position 416 according to SEQ ID NO:32, a cytosine at a position corresponding to position 639 according to SEQ ID NO:33, or a cytosine at a position corresponding to position 600 according to SEQ ID NO:34, and a second primer derived from the 3′ flanking sequence adjacent to a cytosine at a position corresponding to position 176,867 according to SEQ ID NO:2, a cytosine at a position corresponding to position 600 according to SEQ ID NO:11, a cytosine at a position corresponding to position 639 according to SEQ ID NO:12, a cytosine at a position corresponding to position 562 according to SEQ ID NO:13, a cytosine at a position corresponding to position 515 according to SEQ ID NO:14, a cytosine at a position corresponding to position 579 according to SEQ ID NO:15, a cytosine at a position corresponding to position 416 according to SEQ ID NO:16, a cytosine at a position corresponding to position 639 according to SEQ ID NO:17, a cytosine at a position corresponding to position 600 according to SEQ ID NO:18, a cytosine at a position corresponding to position 600 according to SEQ ID NO:27, a cytosine at a position corresponding to position 639 according to SEQ ID NO:28, a cytosine at a position corresponding to position 562 according to SEQ ID NO:29, a cytosine at a position corresponding to position 515 according to SEQ ID NO:30, a cytosine at a position corresponding to position 579 according to SEQ ID NO:31, a cytosine at a position corresponding to position 416 according to SEQ ID NO:32, a cytosine at a position corresponding to position 639 according to SEQ ID NO:33, or a cytosine at a position corresponding to position 600 according to SEQ ID NO:34 to produce an amplicon that is indicative of the presence of the SNP at positions encoding a cytosine at a position corresponding to position 176,867 according to SEQ ID NO:2, a cytosine at a position corresponding to position 600 according to SEQ ID NO:11, a cytosine at a position corresponding to position 639 according to SEQ ID NO:12, a cytosine at a position corresponding to position 562 according to SEQ ID NO:13, a cytosine at a position corresponding to position 515 according to SEQ ID NO:14, a cytosine at a position corresponding to position 579 according to SEQ ID NO:15, a cytosine at a position corresponding to position 416 according to SEQ ID NO:16, a cytosine at a position corresponding to position 639 according to SEQ ID NO:17, a cytosine at a position corresponding to position 600 according to SEQ ID NO:18, a cytosine at a position corresponding to position 600 according to SEQ ID NO:27, a cytosine at a position corresponding to position 639 according to SEQ ID NO:28, a cytosine at a position corresponding to position 562 according to SEQ ID NO:29, a cytosine at a position corresponding to position 515 according to SEQ ID NO:30, a cytosine at a position corresponding to position 579 according to SEQ ID NO:31, a cytosine at a position corresponding to position 416 according to SEQ ID NO:32, a cytosine at a position corresponding to position 639 according to SEQ ID NO:33, or a cytosine at a position corresponding to position 600 according to SEQ ID NO:34. In some embodiments, the amplicon may range in length from the combined length of the primer pairs plus one nucleotide base pair to any length of amplicon producible by a DNA amplification protocol. This distance can range from one nucleotide base pair up to the limits of the amplification reaction, or about twenty thousand nucleotide base pairs. Optionally, the primer pair flanks a region including positions comprising a cytosine at a position corresponding to position 176,867 according to SEQ ID NO:2, a cytosine at a position corresponding to position 600 according to SEQ ID NO:11, a cytosine at a position corresponding to position 639 according to SEQ ID NO:12, a cytosine at a position corresponding to position 562 according to SEQ ID NO:13, a cytosine at a position corresponding to position 515 according to SEQ ID NO:14, a cytosine at a position corresponding to position 579 according to SEQ ID NO:15, a cytosine at a position corresponding to position 416 according to SEQ ID NO:16, a cytosine at a position corresponding to position 639 according to SEQ ID NO:17, a cytosine at a position corresponding to position 600 according to SEQ ID NO:18, a cytosine at a position corresponding to position 600 according to SEQ ID NO:27, a cytosine at a position corresponding to position 639 according to SEQ ID NO:28, a cytosine at a position corresponding to position 562 according to SEQ ID NO:29, a cytosine at a position corresponding to position 515 according to SEQ ID NO:30, a cytosine at a position corresponding to position 579 according to SEQ ID NO:31, a cytosine at a position corresponding to position 416 according to SEQ ID NO:32, a cytosine at a position corresponding to position 639 according to SEQ ID NO:33, or a cytosine at a position corresponding to position 600 according to SEQ ID NO:34 and at least 1, 2, 3, 4, 5, 6, 7, 8, 9, 10, or more nucleotides on each side of positions comprising a cytosine at a position corresponding to position 176,867 according to SEQ ID NO:2, a cytosine at a position corresponding to position 600 according to SEQ ID NO:11, a cytosine at a position corresponding to position 639 according to SEQ ID NO:12, a cytosine at a position corresponding to position 562 according to SEQ ID NO:13, a cytosine at a position corresponding to position 515 according to SEQ ID NO:14, a cytosine at a position corresponding to position 579 according to SEQ ID NO:15, a cytosine at a position corresponding to position 416 according to SEQ ID NO:16, a cytosine at a position corresponding to position 639 according to SEQ ID NO:17, a cytosine at a position corresponding to position 600 according to SEQ ID NO:18, a cytosine at a position corresponding to position 600 according to SEQ ID NO:27, a cytosine at a position corresponding to position 639 according to SEQ ID NO:28, a cytosine at a position corresponding to position 562 according to SEQ ID NO:29, a cytosine at a position corresponding to position 515 according to SEQ ID NO:30, a cytosine at a position corresponding to position 579 according to SEQ ID NO:31, a cytosine at a position corresponding to position 416 according to SEQ ID NO:32, a cytosine at a position corresponding to position 639 according to SEQ ID NO:33, or a cytosine at a position corresponding to position 600 according to SEQ ID NO:34.

Similar amplicons can be generated from the mRNA and/or cDNA sequences. PCR primer pairs can be derived from a known sequence, for example, by using computer programs intended for that purpose, such as the PCR primer analysis tool in Vector NTI version 10 (Informax Inc., Bethesda Md.); PrimerSelect (DNASTAR Inc., Madison, Wis.); and Primer3 (Version 0.4.0.COPYRGT., 1991, Whitehead Institute for Biomedical Research, Cambridge, Mass.). Additionally, the sequence can be visually scanned and primers manually identified using known guidelines.

Illustrative examples of nucleic acid sequencing techniques include, but are not limited to, chain terminator (Sanger) sequencing and dye terminator sequencing. Other methods involve nucleic acid hybridization methods other than sequencing, including using labeled primers or probes directed against purified DNA, amplified DNA, and fixed cell preparations (fluorescence in situ hybridization (FISH)). In some methods, a target nucleic acid molecule may be amplified prior to or simultaneous with detection. Illustrative examples of nucleic acid amplification techniques include, but are not limited to, polymerase chain reaction (PCR), ligase chain reaction (LCR), strand displacement amplification (SDA), and nucleic acid sequence based amplification (NASBA). Other methods include, but are not limited to, ligase chain reaction, strand displacement amplification, and thermophilic SDA (tSDA).

In hybridization techniques, stringent conditions can be employed such that a probe or primer will specifically hybridize to its target. In some embodiments, a polynucleotide primer or probe under stringent conditions will hybridize to its target sequence to a detectably greater degree than to other non-target sequences, such as, at least 2-fold, at least 3-fold, at least 4-fold, or more over background, including over 10-fold over background. In some embodiments, a polynucleotide primer or probe under stringent conditions will hybridize to its target nucleotide sequence to a detectably greater degree than to other nucleotide sequences by at least 2-fold. In some embodiments, a polynucleotide primer or probe under stringent conditions will hybridize to its target nucleotide sequence to a detectably greater degree than to other nucleotide sequences by at least 3-fold. In some embodiments, a polynucleotide primer or probe under stringent conditions will hybridize to its target nucleotide sequence to a detectably greater degree than to other nucleotide sequences by at least 4-fold. In some embodiments, a polynucleotide primer or probe under stringent conditions will hybridize to its target nucleotide sequence to a detectably greater degree than to other nucleotide sequences by over 10-fold over background. Stringent conditions are sequence-dependent and will be different in different circumstances.

Appropriate stringency conditions which promote DNA hybridization, for example, 6× sodium chloride/sodium citrate (SSC) at about 45° C., followed by a wash of 2×SSC at 50° C., are known or can be found in Current Protocols in Molecular Biology, John Wiley & Sons, N.Y. (1989), 6.3.1-6.3.6. Typically, stringent conditions for hybridization and detection will be those in which the salt concentration is less than about 1.5 M Na⁺ ion, typically about 0.01 to 1.0 M Na⁺ ion concentration (or other salts) at pH 7.0 to 8.3 and the temperature is at least about 30° C. for short probes (such as, for example, 10 to 50 nucleotides) and at least about 60° C. for longer probes (such as, for example, greater than 50 nucleotides). Stringent conditions may also be achieved with the addition of destabilizing agents such as formamide. Optionally, wash buffers may comprise about 0.1% to about 1% SDS. Duration of hybridization is generally less than about 24 hours, usually about 4 to about 12 hours. The duration of the wash time will be at least a length of time sufficient to reach equilibrium.

The present disclosure also provides methods of detecting the presence of an ST6GALNAC5 predicted loss-of-function polypeptide comprising performing an assay on a biological sample obtained from the subject to determine whether an ST6GALNAC5 polypeptide in the biological sample contains one or more variations that causes the polypeptide to have a loss-of-function (partial or complete) or predicted loss-of-function (partial or complete). The ST6GALNAC5 predicted loss-of-function polypeptide can be any of the ST6GALNAC5 predicted loss-of-function polypeptides described herein. In some embodiments, the methods detect the presence of ST6GALNAC5 Val135Ala, Val45Ala, Val45A1a*, or Val135A1a*. In some embodiments, the methods detect the presence of ST6GALNAC5 Val135Ala. In some embodiments, the methods detect the presence of ST6GALNAC5 Val45Ala. In some embodiments, the methods detect the presence of ST6GALNAC5 Val45A1a*. In some embodiments, the methods detect the presence of ST6GALNAC5 Val135A1a*.

In some embodiments, the methods comprise performing an assay on a biological sample obtained from a subject to determine whether an ST6GALNAC5 polypeptide in the biological sample comprises an alanine at a position corresponding to position 135 according to SEQ ID NO:39, an alanine at a position corresponding to position 45 according to SEQ ID NO:40, an alanine at a position corresponding to position 45 according to SEQ ID NO:41, or an alanine at a position corresponding to position 135 according to SEQ ID NO:42.

In some embodiments, the detecting step comprises sequencing at least a portion of the ST6GALNAC5 polypeptide that comprises a position corresponding to: position 135 according to SEQ ID NO:39, position 45 according to SEQ ID NO:40, position 45 according to SEQ ID NO:41, or position 135 according to SEQ ID NO:42, or SEQ ID NO:35, SEQ ID NO:36, SEQ ID NO:37, or SEQ ID NO:38.

In some embodiments, the detecting step comprises an immunoassay for detecting the presence of a ST6GALNAC5 polypeptide that comprises a position corresponding to: position 135 according to SEQ ID NO:39, position 45 according to SEQ ID NO:40, position 45 according to SEQ ID NO:41, or position 135 according to SEQ ID NO:42, or SEQ ID NO:35, SEQ ID NO:36, SEQ ID NO:37, or SEQ ID NO:38.

In some embodiments, when the subject does not have an ST6GALNAC5 predicted loss-of-function polypeptide, the subject has an increased risk of developing cognitive impairment or any of decreased myelin integrity, neurodegeneration, decreased GWC, and conversion of mild cognitive impairment to dementia. In some embodiments, when the subject has an ST6GALNAC5 predicted loss-of-function polypeptide, the subject has a decreased risk of developing cognitive impairment or any of decreased myelin integrity, neurodegeneration, decreased GWC, and conversion of mild cognitive impairment to dementia.

The present disclosure also provides isolated nucleic acid molecules that hybridize to ST6GALNAC5 missense variant genomic nucleic acid molecules, ST6GALNAC5 missense variant mRNA molecules, and/or ST6GALNAC5 missense variant cDNA molecules (such as any of the genomic variant nucleic acid molecules, mRNA variant molecules, and cDNA variant molecules disclosed herein). In some embodiments, such isolated nucleic acid molecules hybridize to ST6GALNAC5 missense variant nucleic acid molecules under stringent conditions. Such nucleic acid molecules can be used, for example, as probes, primers, alteration-specific probes, or alteration-specific primers as described or exemplified herein.

In some embodiments, the isolated nucleic acid molecules hybridize to a portion of the ST6GALNAC5 missense nucleic acid molecule that includes a position corresponding to: position 176,867 according to SEQ ID NO:2, position 600 according to SEQ ID NO:11, position 639 according to SEQ ID NO:12, position 562 according to SEQ ID NO:13, position 515 according to SEQ ID NO:14, position 579 according to SEQ ID NO:15, position 416 according to SEQ ID NO:16, position 639 according to SEQ ID NO:17, position 600 according to SEQ ID NO:18, position 600 according to SEQ ID NO:27, position 639 according to SEQ ID NO:28, position 562 according to SEQ ID NO:29, position 515 according to SEQ ID NO:30, position 579 according to SEQ ID NO:31, position 416 according to SEQ ID NO:32, position 639 according to SEQ ID NO:33, or position 600 according to SEQ ID NO:34.

In some embodiments, such isolated nucleic acid molecules comprise or consist of at least about 5, at least about 8, at least about 10, at least about 11, at least about 12, at least about 13, at least about 14, at least about 15, at least about 16, at least about 17, at least about 18, at least about 19, at least about 20, at least about 21, at least about 22, at least about 23, at least about 24, at least about 25, at least about 30, at least about 35, at least about 40, at least about 45, at least about 50, at least about 55, at least about 60, at least about 65, at least about 70, at least about 75, at least about 80, at least about 85, at least about 90, at least about 95, at least about 100, at least about 200, at least about 300, at least about 400, at least about 500, at least about 600, at least about 700, at least about 800, at least about 900, at least about 1000, at least about 2000, at least about 3000, at least about 4000, or at least about 5000 nucleotides. In some embodiments, such isolated nucleic acid molecules comprise or consist of at least about 5, at least about 8, at least about 10, at least about 11, at least about 12, at least about 13, at least about 14, at least about 15, at least about 16, at least about 17, at least about 18, at least about 19, at least about 20, at least about 21, at least about 22, at least about 23, at least about 24, or at least about 25 nucleotides. In some embodiments, the isolated nucleic acid molecules comprise or consist of at least about 18 nucleotides. In some embodiments, the isolated nucleic acid molecules comprise or consists of at least about 15 nucleotides. In some embodiments, the isolated nucleic acid molecules consist of or comprise from about 10 to about 35, from about 10 to about 30, from about 10 to about 25, from about 12 to about 30, from about 12 to about 28, from about 12 to about 24, from about 15 to about 30, from about 15 to about 25, from about 18 to about 30, from about 18 to about 25, from about 18 to about 24, or from about 18 to about 22 nucleotides. In some embodiments, the isolated nucleic acid molecules consist of or comprise from about 18 to about 30 nucleotides. In some embodiments, the isolated nucleic acid molecules comprise or consist of at least about 15 nucleotides to at least about 35 nucleotides.

In some embodiments, the isolated nucleic acid molecules hybridize to at least about 15 contiguous nucleotides of a nucleic acid molecule that is at least about 70%, at least about 75%, at least about 80%, at least about 85%, at least about 90%, at least about 95%, at least about 96%, at least about 97%, at least about 98%, at least about 99%, or 100% identical to ST6GALNAC5 missense variant genomic nucleic acid molecules, ST6GALNAC5 missense variant mRNA molecules, and/or ST6GALNAC5 missense variant cDNA molecules. In some embodiments, the isolated nucleic acid molecules consist of or comprise from about 15 to about 100 nucleotides, or from about 15 to about 35 nucleotides. In some embodiments, the isolated nucleic acid molecules consist of or comprise from about 15 to about 100 nucleotides. In some embodiments, the isolated nucleic acid molecules consist of or comprise from about 15 to about 35 nucleotides.

In some embodiments, the isolated alteration-specific probes or alteration-specific primers comprise at least about 15 nucleotides, wherein the alteration-specific probe or alteration-specific primer comprises a nucleotide sequence which is complementary to the nucleotide sequence of a portion of an ST6GALNAC5 missense nucleic acid molecule encoding a ST6GALNAC5 predicted loss-of-function polypeptide, or the complement thereof. In some embodiments, the portion comprises a position corresponding to: position 176,867 according to SEQ ID NO:2, or the complement thereof; position 600 according to SEQ ID NO:11, or the complement thereof; position 639 according to SEQ ID NO:12, or the complement thereof; position 562 according to SEQ ID NO:13, or the complement thereof; position 515 according to SEQ ID NO:14, or the complement thereof; position 579 according to SEQ ID NO:15, or the complement thereof; position 416 according to SEQ ID NO:16, or the complement thereof; position 639 according to SEQ ID NO:17, or the complement thereof; position 600 according to SEQ ID NO:18, or the complement thereof; position 600 according to SEQ ID NO:27, or the complement thereof; position 639 according to SEQ ID NO:28, or the complement thereof; position 562 according to SEQ ID NO:29, or the complement thereof; position 515 according to SEQ ID NO:30, or the complement thereof; position 579 according to SEQ ID NO:31, or the complement thereof; position 416 according to SEQ ID NO:32, or the complement thereof; position 639 according to SEQ ID NO:33, or the complement thereof; or position 600 according to SEQ ID NO:34, or the complement thereof. In some embodiments, the portion comprises positions corresponding to: positions 176,866-176,868 according to SEQ ID NO:2, or the complement thereof; positions 599-601 according to SEQ ID NO:11, or the complement thereof; positions 638-640 according to SEQ ID NO:12, or the complement thereof; positions 561-563 according to SEQ ID NO:13, or the complement thereof; positions 514-516 according to SEQ ID NO:14, or the complement thereof; positions 578-580 according to SEQ ID NO:15, or the complement thereof; positions 415-417 according to SEQ ID NO:16, or the complement thereof; positions 638-640 according to SEQ ID NO:17, or the complement thereof; positions 599-601 according to SEQ ID NO:18, or the complement thereof; positions 599-601 according to SEQ ID NO:27, or the complement thereof; positions 638-640 according to SEQ ID NO:28, or the complement thereof; positions 561-563 according to SEQ ID NO:29, or the complement thereof; positions 514-516 according to SEQ ID NO:30, or the complement thereof; positions 578-580 according to SEQ ID NO:31, or the complement thereof; positions 415-417 according to SEQ ID NO:32, or the complement thereof; positions 638-640 according to SEQ ID NO:33, or the complement thereof; or positions 599-601 according to SEQ ID NO:34, or the complement thereof.

In some embodiments, the alteration-specific probes and alteration-specific primers comprise DNA. In some embodiments, the alteration-specific probes and alteration-specific primers comprise RNA.

In some embodiments, the probes and primers described herein (including alteration-specific probes and alteration-specific primers) have a nucleotide sequence that specifically hybridizes to any of the nucleic acid molecules disclosed herein, or the complement thereof. In some embodiments, the probes and primers specifically hybridize to any of the nucleic acid molecules disclosed herein under stringent conditions.

In some embodiments, the primers, including alteration-specific primers, can be used in second generation sequencing or high throughput sequencing. In some instances, the primers, including alteration-specific primers, can be modified. In particular, the primers can comprise various modifications that are used at different steps of, for example, Massive Parallel Signature Sequencing (MPSS), Polony sequencing, and 454 Pyrosequencing. Modified primers can be used at several steps of the process, including biotinylated primers in the cloning step and fluorescently labeled primers used at the bead loading step and detection step. Polony sequencing is generally performed using a paired-end tags library wherein each molecule of DNA template is about 135 bp in length. Biotinylated primers are used at the bead loading step and emulsion PCR. Fluorescently labeled degenerate nonamer oligonucleotides are used at the detection step. An adaptor can contain a 5′-biotin tag for immobilization of the DNA library onto streptavidin-coated beads.

The probes and primers described herein can be used to detect a nucleotide variation within any of the ST6GALNAC5 missense variant genomic nucleic acid molecules, ST6GALNAC5 missense variant mRNA molecules, and/or ST6GALNAC5 missense variant cDNA molecules disclosed herein. The primers described herein can be used to amplify the ST6GALNAC5 missense variant genomic nucleic acid molecules, ST6GALNAC5 missense variant mRNA molecules, or ST6GALNAC5 missense variant cDNA molecules, or a fragment thereof.

The present disclosure also provides pairs of primers comprising any of the primers described above. For example, if one of the primers' 3′-ends hybridizes to a thymine at a position corresponding to position 176,867 according to SEQ ID NO:1 (rather than a cytosine) in a particular ST6GALNAC5 nucleic acid molecule, then the presence of the amplified fragment would indicate the presence of an ST6GALNAC5 reference genomic nucleic acid molecule. Conversely, if one of the primers' 3′-ends hybridizes to a cytosine at a position corresponding to position 176,867 according to SEQ ID NO:2 (rather than a thymine) in a particular ST6GALNAC5 nucleic acid molecule, then the presence of the amplified fragment would indicate the presence of the ST6GALNAC5 missense variant genomic nucleic acid molecule. In some embodiments, the nucleotide of the primer complementary to the cytosine at a position corresponding to position 176,867 according to SEQ ID NO:2 can be at the 3′ end of the primer.

If one of the primers' 3′-ends hybridizes to a uracil at a position corresponding to position 600 according to SEQ ID NO:3 (rather than a cytosine) in a particular ST6GALNAC5 nucleic acid molecule, then the presence of the amplified fragment would indicate the presence of an ST6GALNAC5 reference mRNA molecule. Conversely, if one of the primers' 3′-ends hybridizes to a cytosine at a position corresponding to position 600 according to SEQ ID NO:11 (rather than a uracil) in a particular ST6GALNAC5 mRNA molecule, then the presence of the amplified fragment would indicate the presence of the ST6GALNAC5 missense variant mRNA molecule. In some embodiments, the nucleotide of the primer complementary to the cytosine at a position corresponding to position 600 according to SEQ ID NO:11 can be at the 3′ end of the primer.

If one of the primers' 3′-ends hybridizes to a uracil at a position corresponding to position 639 according to SEQ ID NO:4 (rather than a cytosine) in a particular ST6GALNAC5 nucleic acid molecule, then the presence of the amplified fragment would indicate the presence of an ST6GALNAC5 reference mRNA molecule. Conversely, if one of the primers' 3′-ends hybridizes to a cytosine at a position corresponding to position 639 according to SEQ ID NO:12 (rather than a uracil) in a particular ST6GALNAC5 mRNA molecule, then the presence of the amplified fragment would indicate the presence of the ST6GALNAC5 missense variant mRNA molecule. In some embodiments, the nucleotide of the primer complementary to the cytosine at a position corresponding to position 639 according to SEQ ID NO:12 can be at the 3′ end of the primer.

If one of the primers' 3′-ends hybridizes to a uracil at a position corresponding to position 562 according to SEQ ID NO:5 (rather than a cytosine) in a particular ST6GALNAC5 nucleic acid molecule, then the presence of the amplified fragment would indicate the presence of an ST6GALNAC5 reference mRNA molecule. Conversely, if one of the primers' 3′-ends hybridizes to a cytosine at a position corresponding to position 562 according to SEQ ID NO:13 (rather than a uracil) in a particular ST6GALNAC5 mRNA molecule, then the presence of the amplified fragment would indicate the presence of the ST6GALNAC5 missense variant mRNA molecule. In some embodiments, the nucleotide of the primer complementary to the cytosine at a position corresponding to position 562 according to SEQ ID NO:13 can be at the 3′ end of the primer.

If one of the primers' 3′-ends hybridizes to a uracil at a position corresponding to position 515 according to SEQ ID NO:6 (rather than a cytosine) in a particular ST6GALNAC5 nucleic acid molecule, then the presence of the amplified fragment would indicate the presence of an ST6GALNAC5 reference mRNA molecule. Conversely, if one of the primers' 3′-ends hybridizes to a cytosine at a position corresponding to position 515 according to SEQ ID NO:14 (rather than a uracil) in a particular ST6GALNAC5 mRNA molecule, then the presence of the amplified fragment would indicate the presence of the ST6GALNAC5 missense variant mRNA molecule. In some embodiments, the nucleotide of the primer complementary to the cytosine at a position corresponding to position 515 according to SEQ ID NO:14 can be at the 3′ end of the primer.

If one of the primers' 3′-ends hybridizes to a uracil at a position corresponding to position 579 according to SEQ ID NO:7 (rather than a cytosine) in a particular ST6GALNAC5 nucleic acid molecule, then the presence of the amplified fragment would indicate the presence of an ST6GALNAC5 reference mRNA molecule. Conversely, if one of the primers' 3′-ends hybridizes to a cytosine at a position corresponding to position 579 according to SEQ ID NO:15 (rather than a uracil) in a particular ST6GALNAC5 mRNA molecule, then the presence of the amplified fragment would indicate the presence of the ST6GALNAC5 missense variant mRNA molecule. In some embodiments, the nucleotide of the primer complementary to the cytosine at a position corresponding to position 579 according to SEQ ID NO:15 can be at the 3′ end of the primer.

If one of the primers' 3′-ends hybridizes to a uracil at a position corresponding to position 416 according to SEQ ID NO:8 (rather than a cytosine) in a particular ST6GALNAC5 nucleic acid molecule, then the presence of the amplified fragment would indicate the presence of an ST6GALNAC5 reference mRNA molecule. Conversely, if one of the primers' 3′-ends hybridizes to a cytosine at a position corresponding to position 416 according to SEQ ID NO:16 (rather than a uracil) in a particular ST6GALNAC5 mRNA molecule, then the presence of the amplified fragment would indicate the presence of the ST6GALNAC5 missense variant mRNA molecule. In some embodiments, the nucleotide of the primer complementary to the cytosine at a position corresponding to position 416 according to SEQ ID NO:16 can be at the 3′ end of the primer.

If one of the primers' 3′-ends hybridizes to a uracil at a position corresponding to position 639 according to SEQ ID NO:9 (rather than a cytosine) in a particular ST6GALNAC5 nucleic acid molecule, then the presence of the amplified fragment would indicate the presence of an ST6GALNAC5 reference mRNA molecule. Conversely, if one of the primers' 3′-ends hybridizes to a cytosine at a position corresponding to position 639 according to SEQ ID NO:17 (rather than a uracil) in a particular ST6GALNAC5 mRNA molecule, then the presence of the amplified fragment would indicate the presence of the ST6GALNAC5 missense variant mRNA molecule. In some embodiments, the nucleotide of the primer complementary to the cytosine at a position corresponding to position 639 according to SEQ ID NO:17 can be at the 3′ end of the primer.

If one of the primers' 3′-ends hybridizes to a uracil at a position corresponding to position 600 according to SEQ ID NO:10 (rather than a cytosine) in a particular ST6GALNAC5 nucleic acid molecule, then the presence of the amplified fragment would indicate the presence of an ST6GALNAC5 reference mRNA molecule. Conversely, if one of the primers' 3′-ends hybridizes to a cytosine at a position corresponding to position 600 according to SEQ ID NO:18 (rather than a uracil) in a particular ST6GALNAC5 mRNA molecule, then the presence of the amplified fragment would indicate the presence of the ST6GALNAC5 missense variant mRNA molecule. In some embodiments, the nucleotide of the primer complementary to the cytosine at a position corresponding to position 600 according to SEQ ID NO:18 can be at the 3′ end of the primer.

If one of the primers' 3′-ends hybridizes to a thymine at a position corresponding to position 600 according to SEQ ID NO:19 (rather than a cytosine) in a particular ST6GALNAC5 nucleic acid molecule, then the presence of the amplified fragment would indicate the presence of an ST6GALNAC5 reference cDNA molecule. Conversely, if one of the primers' 3′-ends hybridizes to a cytosine at a position corresponding to position 600 according to SEQ ID NO:27 (rather than a thymine) in a particular ST6GALNAC5 cDNA molecule, then the presence of the amplified fragment would indicate the presence of the ST6GALNAC5 missense variant cDNA molecule. In some embodiments, the nucleotide of the primer complementary to the cytosine at a position corresponding to position 600 according to SEQ ID NO:27 can be at the 3′ end of the primer.

If one of the primers' 3′-ends hybridizes to a thymine at a position corresponding to position 639 according to SEQ ID NO:20 (rather than a cytosine) in a particular ST6GALNAC5 nucleic acid molecule, then the presence of the amplified fragment would indicate the presence of an ST6GALNAC5 reference cDNA molecule. Conversely, if one of the primers' 3′-ends hybridizes to a cytosine at a position corresponding to position 639 according to SEQ ID NO:28 (rather than a thymine) in a particular ST6GALNAC5 cDNA molecule, then the presence of the amplified fragment would indicate the presence of the ST6GALNAC5 missense variant cDNA molecule. In some embodiments, the nucleotide of the primer complementary to the cytosine at a position corresponding to position 639 according to SEQ ID NO:28 can be at the 3′ end of the primer.

If one of the primers' 3′-ends hybridizes to a thymine at a position corresponding to position 562 according to SEQ ID NO:21 (rather than a cytosine) in a particular ST6GALNAC5 nucleic acid molecule, then the presence of the amplified fragment would indicate the presence of an ST6GALNAC5 reference cDNA molecule. Conversely, if one of the primers' 3′-ends hybridizes to a cytosine at a position corresponding to position 562 according to SEQ ID NO:29 (rather than a thymine) in a particular ST6GALNAC5 cDNA molecule, then the presence of the amplified fragment would indicate the presence of the ST6GALNAC5 missense variant cDNA molecule. In some embodiments, the nucleotide of the primer complementary to the cytosine at a position corresponding to position 562 according to SEQ ID NO:29 can be at the 3′ end of the primer.

If one of the primers' 3′-ends hybridizes to a thymine at a position corresponding to position 515 according to SEQ ID NO:22 (rather than a cytosine) in a particular ST6GALNAC5 nucleic acid molecule, then the presence of the amplified fragment would indicate the presence of an ST6GALNAC5 reference cDNA molecule. Conversely, if one of the primers' 3′-ends hybridizes to a cytosine at a position corresponding to position 515 according to SEQ ID NO:30 (rather than a thymine) in a particular ST6GALNAC5 cDNA molecule, then the presence of the amplified fragment would indicate the presence of the ST6GALNAC5 missense variant cDNA molecule. In some embodiments, the nucleotide of the primer complementary to the cytosine at a position corresponding to position 515 according to SEQ ID NO:30 can be at the 3′ end of the primer.

If one of the primers' 3′-ends hybridizes to a thymine at a position corresponding to position 579 according to SEQ ID NO:23 (rather than a cytosine) in a particular ST6GALNAC5 nucleic acid molecule, then the presence of the amplified fragment would indicate the presence of an ST6GALNAC5 reference cDNA molecule. Conversely, if one of the primers' 3′-ends hybridizes to a cytosine at a position corresponding to position 579 according to SEQ ID NO:31 (rather than a thymine) in a particular ST6GALNAC5 cDNA molecule, then the presence of the amplified fragment would indicate the presence of the ST6GALNAC5 missense variant cDNA molecule. In some embodiments, the nucleotide of the primer complementary to the cytosine at a position corresponding to position 579 according to SEQ ID NO:31 can be at the 3′ end of the primer.

If one of the primers' 3′-ends hybridizes to a thymine at a position corresponding to position 416 according to SEQ ID NO:24 (rather than a cytosine) in a particular ST6GALNAC5 nucleic acid molecule, then the presence of the amplified fragment would indicate the presence of an ST6GALNAC5 reference cDNA molecule. Conversely, if one of the primers' 3′-ends hybridizes to a cytosine at a position corresponding to position 416 according to SEQ ID NO:32 (rather than a thymine) in a particular ST6GALNAC5 cDNA molecule, then the presence of the amplified fragment would indicate the presence of the ST6GALNAC5 missense variant cDNA molecule. In some embodiments, the nucleotide of the primer complementary to the cytosine at a position corresponding to position 416 according to SEQ ID NO:32 can be at the 3′ end of the primer.

If one of the primers' 3′-ends hybridizes to a thymine at a position corresponding to position 639 according to SEQ ID NO:25 (rather than a cytosine) in a particular ST6GALNAC5 nucleic acid molecule, then the presence of the amplified fragment would indicate the presence of an ST6GALNAC5 reference cDNA molecule. Conversely, if one of the primers' 3′-ends hybridizes to a cytosine at a position corresponding to position 639 according to SEQ ID NO:33 (rather than a thymine) in a particular ST6GALNAC5 cDNA molecule, then the presence of the amplified fragment would indicate the presence of the ST6GALNAC5 missense variant cDNA molecule. In some embodiments, the nucleotide of the primer complementary to the cytosine at a position corresponding to position 639 according to SEQ ID NO:33 can be at the 3′ end of the primer.

If one of the primers' 3′-ends hybridizes to a thymine at a position corresponding to position 600 according to SEQ ID NO:26 (rather than a cytosine) in a particular ST6GALNAC5 nucleic acid molecule, then the presence of the amplified fragment would indicate the presence of an ST6GALNAC5 reference cDNA molecule. Conversely, if one of the primers' 3′-ends hybridizes to a cytosine at a position corresponding to position 600 according to SEQ ID NO:34 (rather than a thymine) in a particular ST6GALNAC5 cDNA molecule, then the presence of the amplified fragment would indicate the presence of the ST6GALNAC5 missense variant cDNA molecule. In some embodiments, the nucleotide of the primer complementary to the cytosine at a position corresponding to position 600 according to SEQ ID NO:34 can be at the 3′ end of the primer.

In the context of the present disclosure “specifically hybridizes” means that the probe or primer (such as, for example, the alteration-specific probe or alteration-specific primer) does not hybridize to a nucleic acid sequence encoding an ST6GALNAC5 reference genomic nucleic acid molecule, an ST6GALNAC5 reference mRNA molecule, and/or an ST6GALNAC5 reference cDNA molecule.

In any of the embodiments described throughout the present disclosure, the probes (such as, for example, an alteration-specific probe) can comprise a label. In some embodiments, the label is a fluorescent label, a radiolabel, or biotin.

The present disclosure also provides supports comprising a substrate to which any one or more of the probes disclosed herein is attached. Solid supports are solid-state substrates or supports with which molecules, such as any of the probes disclosed herein, can be associated. A form of solid support is an array. Another form of solid support is an array detector. An array detector is a solid support to which multiple different probes have been coupled in an array, grid, or other organized pattern. A form for a solid-state substrate is a microtiter dish, such as a standard 96-well type. In some embodiments, a multiwell glass slide can be employed that normally contains one array per well. In some embodiments, the support is a microarray.

The present disclosure also provides molecular complexes comprising or consisting of any of the ST6GALNAC5 missense nucleic acid molecules (genomic nucleic acid molecules, mRNA molecules, or cDNA molecules), or complement thereof, described herein and any of the alteration-specific primers or alteration-specific probes described herein. In some embodiments, the ST6GALNAC5 missense nucleic acid molecules (genomic nucleic acid molecules, mRNA molecules, or cDNA molecules), or complement thereof, in the molecular complexes are single-stranded. In some embodiments, the ST6GALNAC5 missense nucleic acid molecule is any of the genomic nucleic acid molecules described herein. In some embodiments, the ST6GALNAC5 missense nucleic acid molecule is any of the mRNA molecules described herein. In some embodiments, the ST6GALNAC5 missense nucleic acid molecule is any of the cDNA molecules described herein. In some embodiments, the molecular complex comprises or consists of any of the ST6GALNAC5 missense nucleic acid molecules (genomic nucleic acid molecules, mRNA molecules, or cDNA molecules), or complement thereof, described herein and any of the alteration-specific primers described herein. In some embodiments, the molecular complex comprises or consists of any of the ST6GALNAC5 missense nucleic acid molecules (genomic nucleic acid molecules, mRNA molecules, or cDNA molecules), or complement thereof, described herein and any of the alteration-specific probes described herein.

In some embodiments, the molecular complex comprises or consists of an alteration-specific primer or an alteration-specific probe hybridized to an ST6GALNAC5 genomic nucleic acid molecule encoding an ST6GALNAC5 predicted loss-of-function polypeptide, wherein the alteration-specific primer or the alteration-specific probe is hybridized to the ST6GALNAC5 genomic nucleic acid molecule at a position corresponding to: position 176,867 according to SEQ ID NO:2, or the complement thereof.

In some embodiments, the molecular complex comprises or consists of an alteration-specific primer or an alteration-specific probe that is hybridized to a GCG codon at positions corresponding to positions 176,866-176,868 according to SEQ ID NO:2.

In some embodiments, the molecular complex comprises or consists of a genomic nucleic acid molecule that comprises SEQ ID NO:2.

In some embodiments, the molecular complex comprises or consists of an alteration-specific primer or an alteration-specific probe hybridized to an ST6GALNAC5 mRNA molecule encoding an ST6GALNAC5 predicted loss-of-function polypeptide, wherein the alteration-specific primer or the alteration-specific probe is hybridized to the ST6GALNAC5 mRNA molecule at a position corresponding to: position 600 according to SEQ ID NO:11, or the complement thereof; position 639 according to SEQ ID NO:12, or the complement thereof; position 562 according to SEQ ID NO:13, or the complement thereof; position 515 according to SEQ ID NO:14, or the complement thereof; position 579 according to SEQ ID NO:15, or the complement thereof; position 416 according to SEQ ID NO:16, or the complement thereof; position 639 according to SEQ ID NO:17, or the complement thereof; or position 600 according to SEQ ID NO:18, or the complement thereof.

In some embodiments, the molecular complex comprises or consists of an alteration-specific primer or an alteration-specific probe that is hybridized to: a GCG codon at positions corresponding to positions 599-601 according to SEQ ID NO:11, a GCG codon at positions corresponding to positions 638-640 according to SEQ ID NO:12, a GCG codon at positions corresponding to positions 561-563 according to SEQ ID NO:13, a GCG codon at positions corresponding to positions 514-516 according to SEQ ID NO:14, a GCG codon at positions corresponding to positions 578-580 according to SEQ ID NO:15, a GCG codon at positions corresponding to positions 415-417 according to SEQ ID NO:16, a GCG codon at positions corresponding to positions 638-640 according to SEQ ID NO:17, or a GCG codon at positions corresponding to positions 599-601 according to SEQ ID NO:18.

In some embodiments, the molecular complex comprises or consists of an mRNA molecule that comprises SEQ ID NO:11, SEQ ID NO:12, SEQ ID NO:13, SEQ ID NO:14, SEQ ID NO:15, SEQ ID NO:16, SEQ ID NO:17, or SEQ ID NO:18.

In some embodiments, the molecular complex comprises or consists of an alteration-specific primer or an alteration-specific probe hybridized to an ST6GALNAC5 cDNA molecule encoding an ST6GALNAC5 predicted loss-of-function polypeptide, wherein the alteration-specific primer or the alteration-specific probe is hybridized to the ST6GALNAC5 cDNA molecule at a position corresponding to: position 600 according to SEQ ID NO:27, or the complement thereof; position 639 according to SEQ ID NO:28, or the complement thereof; position 562 according to SEQ ID NO:29, or the complement thereof; position 515 according to SEQ ID NO:30, or the complement thereof; position 579 according to SEQ ID NO:31, or the complement thereof; position 416 according to SEQ ID NO:32, or the complement thereof; position 639 according to SEQ ID NO:33, or the complement thereof; or position 600 according to SEQ ID NO:34, or the complement thereof.

In some embodiments, the molecular complex comprises or consists of an alteration-specific primer or an alteration-specific probe that is hybridized to: a GCG codon at positions corresponding to positions 599-601 according to SEQ ID NO:27, a GCG codon at positions corresponding to positions 638-640 according to SEQ ID NO:28, a GCG codon at positions corresponding to positions 561-563 according to SEQ ID NO:29, a GCG codon at positions corresponding to positions 514-516 according to SEQ ID NO:30, a GCG codon at positions corresponding to positions 578-580 according to SEQ ID NO:31, a GCG codon at positions corresponding to positions 415-417 according to SEQ ID NO:32, a GCG codon at positions corresponding to positions 638-640 according to SEQ ID NO:33, or a GCG codon at positions corresponding to positions 599-601 according to SEQ ID NO:34.

In some embodiments, the molecular complex comprises or consists of an cDNA molecule that comprises SEQ ID NO:27, SEQ ID NO:28, SEQ ID NO:29, SEQ ID NO:30, SEQ ID NO:31, SEQ ID NO:32, SEQ ID NO:33, or SEQ ID NO:34.

In some embodiments, the molecular complex comprises an alteration-specific probe or an alteration-specific primer comprising a label. In some embodiments, the label is a fluorescent label, a radiolabel, or biotin. In some embodiments, the molecular complex further comprises a non-human polymerase.

The nucleotide sequence of an ST6GALNAC5 reference genomic nucleic acid molecule is set forth in SEQ ID NO:1. Referring to SEQ ID NO:1, position 176,867 is a thymine.

A ST6GALNAC5 missense variant genomic nucleic acid molecule exists, wherein the thymine at position 176,867 is replaced with a cytosine (r5756654226; chr1:77044346 in the GRCh38/hg38 human genome assembly). The nucleotide sequence of this ST6GALNAC5 missense variant genomic nucleic acid molecule is set forth in SEQ ID NO:2.

The nucleotide sequence of an ST6GALNAC5 reference mRNA molecule is set forth in SEQ ID NO:3. Referring to SEQ ID NO:3, position 600 is a uracil. The nucleotide sequence of another ST6GALNAC5 reference mRNA molecule is set forth in SEQ ID NO:4. Referring to SEQ ID NO:4, position 639 is a uracil. The nucleotide sequence of another ST6GALNAC5 reference mRNA molecule is set forth in SEQ ID NO:5. Referring to SEQ ID NO:5, position 562 is a uracil. The nucleotide sequence of another ST6GALNAC5 reference mRNA molecule is set forth in SEQ ID NO:6. Referring to SEQ ID NO:6, position 515 is a uracil. The nucleotide sequence of another ST6GALNAC5 reference mRNA molecule is set forth in SEQ ID NO:7. Referring to SEQ ID NO:7, position 579 is a uracil. The nucleotide sequence of another ST6GALNAC5 reference mRNA molecule is set forth in SEQ ID NO:8. Referring to SEQ ID NO:8, position 416 is a uracil. The nucleotide sequence of another ST6GALNAC5 reference mRNA molecule is set forth in SEQ ID NO:9. Referring to SEQ ID NO:9, position 639 is a uracil. The nucleotide sequence of another ST6GALNAC5 reference mRNA molecule is set forth in SEQ ID NO:10. Referring to SEQ ID NO:10, position 600 is a uracil.

A ST6GALNAC5 missense variant mRNA molecule exists, wherein the uracil at position 600 is replaced with a cytosine. The nucleotide sequence of this ST6GALNAC5 missense variant mRNA molecule is set forth in SEQ ID NO:11.

Another ST6GALNAC5 missense variant mRNA molecule exists, wherein the uracil at position 639 is replaced with a cytosine. The nucleotide sequence of this ST6GALNAC5 missense variant mRNA molecule is set forth in SEQ ID NO:12.

Another ST6GALNAC5 missense variant mRNA molecule exists, wherein the uracil at position 562 is replaced with a cytosine. The nucleotide sequence of this ST6GALNAC5 missense variant mRNA molecule is set forth in SEQ ID NO:13.

Another ST6GALNAC5 missense variant mRNA molecule exists, wherein the uracil at position 515 is replaced with a cytosine. The nucleotide sequence of this ST6GALNAC5 missense variant mRNA molecule is set forth in SEQ ID NO:14.

Another ST6GALNAC5 missense variant mRNA molecule exists, wherein the uracil at position 579 is replaced with a cytosine. The nucleotide sequence of this ST6GALNAC5 missense variant mRNA molecule is set forth in SEQ ID NO:15.

Another ST6GALNAC5 missense variant mRNA molecule exists, wherein the uracil at position 416 is replaced with a cytosine. The nucleotide sequence of this ST6GALNAC5 missense variant mRNA molecule is set forth in SEQ ID NO:16.

Another ST6GALNAC5 missense variant mRNA molecule exists, wherein the uracil at position 639 is replaced with a cytosine. The nucleotide sequence of this ST6GALNAC5 missense variant mRNA molecule is set forth in SEQ ID NO:17.

Another ST6GALNAC5 missense variant mRNA molecule exists, wherein the uracil at position 600 is replaced with a cytosine. The nucleotide sequence of this ST6GALNAC5 missense variant mRNA molecule is set forth in SEQ ID NO:18.

The nucleotide sequence of an ST6GALNAC5 reference cDNA molecule is set forth in SEQ ID NO:19. Referring to SEQ ID NO:19, position 600 is a thymine. The nucleotide sequence of another ST6GALNAC5 reference cDNA molecule is set forth in SEQ ID NO:20. Referring to SEQ ID NO:20, position 639 is a thymine. The nucleotide sequence of another ST6GALNAC5 reference cDNA molecule is set forth in SEQ ID NO:21. Referring to SEQ ID NO:21, position 562 is a thymine. The nucleotide sequence of another ST6GALNAC5 reference cDNA molecule is set forth in SEQ ID NO:22. Referring to SEQ ID NO:22, position 515 is a thymine. The nucleotide sequence of another ST6GALNAC5 reference cDNA molecule is set forth in SEQ ID NO:23. Referring to SEQ ID NO:23, position 579 is a thymine. The nucleotide sequence of another ST6GALNAC5 reference cDNA molecule is set forth in SEQ ID NO:24. Referring to SEQ ID NO:24, position 416 is a thymine. The nucleotide sequence of another ST6GALNAC5 reference cDNA molecule is set forth in SEQ ID NO:25. Referring to SEQ ID NO:25, position 639 is a thymine. The nucleotide sequence of another ST6GALNAC5 reference cDNA molecule is set forth in SEQ ID NO:26. Referring to SEQ ID NO:26, position 600 is a thymine.

A ST6GALNAC5 missense variant cDNA molecule exists, wherein the thymine at position 600 is replaced with a cytosine. The nucleotide sequence of this ST6GALNAC5 missense variant cDNA molecule is set forth in SEQ ID NO:27.

Another ST6GALNAC5 missense variant cDNA molecule exists, wherein the thymine at position 639 is replaced with a cytosine. The nucleotide sequence of this ST6GALNAC5 missense variant cDNA molecule is set forth in SEQ ID NO:28.

Another ST6GALNAC5 missense variant cDNA molecule exists, wherein the thymine at position 562 is replaced with a cytosine. The nucleotide sequence of this ST6GALNAC5 missense variant cDNA molecule is set forth in SEQ ID NO:29.

Another ST6GALNAC5 missense variant cDNA molecule exists, wherein the thymine at position 515 is replaced with a cytosine. The nucleotide sequence of this ST6GALNAC5 missense variant cDNA molecule is set forth in SEQ ID NO:30.

Another ST6GALNAC5 missense variant cDNA molecule exists, wherein the thymine at position 579 is replaced with a cytosine. The nucleotide sequence of this ST6GALNAC5 missense variant cDNA molecule is set forth in SEQ ID NO:31.

Another ST6GALNAC5 missense variant cDNA molecule exists, wherein the thymine at position 416 is replaced with a cytosine. The nucleotide sequence of this ST6GALNAC5 missense variant cDNA molecule is set forth in SEQ ID NO:32.

Another ST6GALNAC5 missense variant cDNA molecule exists, wherein the thymine at position 639 is replaced with a cytosine. The nucleotide sequence of this ST6GALNAC5 missense variant cDNA molecule is set forth in SEQ ID NO:33.

Another ST6GALNAC5 missense variant cDNA molecule exists, wherein the thymine at position 600 is replaced with a cytosine. The nucleotide sequence of this ST6GALNAC5 missense variant cDNA molecule is set forth in SEQ ID NO:34.

The genomic nucleic acid molecules, mRNA molecules, and cDNA molecules can be from any organism. For example, the genomic nucleic acid molecules, mRNA molecules, and cDNA molecules can be human or an ortholog from another organism, such as a non-human mammal, a rodent, a mouse, or a rat. It is understood that gene sequences within a population can vary due to polymorphisms such as single-nucleotide polymorphisms. The examples provided herein are only exemplary sequences. Other sequences are also possible.

Also provided herein are functional polynucleotides that can interact with the disclosed nucleic acid molecules. Examples of functional polynucleotides include, but are not limited to, antisense molecules, aptamers, ribozymes, triplex forming molecules, and external guide sequences. The functional polynucleotides can act as effectors, inhibitors, modulators, and stimulators of a specific activity possessed by a target molecule, or the functional polynucleotides can possess a de novo activity independent of any other molecules.

The isolated nucleic acid molecules disclosed herein can comprise RNA, DNA, or both RNA and DNA. The isolated nucleic acid molecules can also be linked or fused to a heterologous nucleic acid sequence, such as in a vector, or a heterologous label. For example, the isolated nucleic acid molecules disclosed herein can be within a vector or as an exogenous donor sequence comprising the isolated nucleic acid molecule and a heterologous nucleic acid sequence. The isolated nucleic acid molecules can also be linked or fused to a heterologous label. The label can be directly detectable (such as, for example, fluorophore) or indirectly detectable (such as, for example, hapten, enzyme, or fluorophore quencher). Such labels can be detectable by spectroscopic, photochemical, biochemical, immunochemical, or chemical means. Such labels include, for example, radiolabels, pigments, dyes, chromogens, spin labels, and fluorescent labels. The label can also be, for example, a chemiluminescent substance; a metal-containing substance; or an enzyme, where there occurs an enzyme-dependent secondary generation of signal. The term “label” can also refer to a “tag” or hapten that can bind selectively to a conjugated molecule such that the conjugated molecule, when added subsequently along with a substrate, is used to generate a detectable signal. For example, biotin can be used as a tag along with an avidin or streptavidin conjugate of horseradish peroxidate (HRP) to bind to the tag, and examined using a calorimetric substrate (such as, for example, tetramethylbenzidine (TMB)) or a fluorogenic substrate to detect the presence of HRP. Exemplary labels that can be used as tags to facilitate purification include, but are not limited to, myc, HA, FLAG or 3×FLAG, 6×His or polyhistidine, glutathione-S-transferase (GST), maltose binding protein, an epitope tag, or the Fc portion of immunoglobulin. Numerous labels include, for example, particles, fluorophores, haptens, enzymes and their calorimetric, fluorogenic and chemiluminescent substrates and other labels.

The isolated nucleic acid molecules, or the complement thereof, can also be present within a host cell. In some embodiments, the host cell can comprise the vector that comprises any of the nucleic acid molecules described herein, or the complement thereof. In some embodiments, the nucleic acid molecule is operably linked to a promoter active in the host cell. In some embodiments, the promoter is an exogenous promoter. In some embodiments, the promoter is an inducible promoter. In some embodiments, the host cell is a bacterial cell, a yeast cell, an insect cell, or a mammalian cell. In some embodiments, the host cell is a bacterial cell. In some embodiments, the host cell is a yeast cell. In some embodiments, the host cell is an insect cell. In some embodiments, the host cell is a mammalian cell.

The disclosed nucleic acid molecules can comprise, for example, nucleotides or non-natural or modified nucleotides, such as nucleotide analogs or nucleotide substitutes. Such nucleotides include a nucleotide that contains a modified base, sugar, or phosphate group, or that incorporates a non-natural moiety in its structure. Examples of non-natural nucleotides include, but are not limited to, dideoxynucleotides, biotinylated, aminated, deaminated, alkylated, benzylated, and fluorophor-labeled nucleotides.

The nucleic acid molecules disclosed herein can also comprise one or more nucleotide analogs or substitutions. A nucleotide analog is a nucleotide which contains a modification to either the base, sugar, or phosphate moieties. Modifications to the base moiety include, but are not limited to, natural and synthetic modifications of A, C, G, and T/U, as well as different purine or pyrimidine bases such as, for example, pseudouridine, uracil-5-yl, hypoxanthin-9-yl (I), and 2-aminoadenin-9-yl. Modified bases include, but are not limited to, 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl uracil and cytosine, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo (such as, for example, 5-bromo), 5-trifluoromethyl and other 5-substituted uracils and cytosines, 7-methylguanine, 7-methyladenine, 8-azaguanine, 8-azaadenine, 7-deazaguanine, 7-deazaadenine, 3-deazaguanine, and 3-deazaadenine.

Nucleotide analogs can also include modifications of the sugar moiety. Modifications to the sugar moiety include, but are not limited to, natural modifications of the ribose and deoxy ribose as well as synthetic modifications. Sugar modifications include, but are not limited to, the following modifications at the 2′ position: OH; F; O-, S-, or N-alkyl; O-, S-, or N-alkenyl; O-, S- or N-alkynyl; or O-alkyl-O-alkyl, wherein the alkyl, alkenyl, and alkynyl may be substituted or unsubstituted C₁₋₁₀alkyl or C₂₋₁₀alkenyl, and C₂₋₁₀alkynyl. Exemplary 2′ sugar modifications also include, but are not limited to, —O[(CH₂)ₙO]ₘCH₃, —O(CH₂)ₙOCH₃, —O(CH₂)ₙNH₂, —O(CH₂)ₙCH₃, —O(CH₂)ₙ—ONH₂, and —O(CH₂)ₙON[(CH₂)ₙCH₃)]₂, where n and m, independently, are from 1 to about 10. Other modifications at the 2′ position include, but are not limited to, C₁₋₁₀alkyl, substituted lower alkyl, alkaryl, aralkyl, O-alkaryl or O-aralkyl, SH, SCH₃, OCN, Cl, Br, CN, CF₃, OCF₃, SOCH₃, SO₂CH₃, ONO₂, NO₂, N₃, NH₂, heterocycloalkyl, heterocycloalkaryl, aminoalkylamino, polyalkylamino, substituted silyl, an RNA cleaving group, a reporter group, an intercalator, a group for improving the pharmacokinetic properties of an oligonucleotide, or a group for improving the pharmacodynamic properties of an oligonucleotide, and other substituents having similar properties. Similar modifications may also be made at other positions on the sugar, particularly the 3′ position of the sugar on the 3′ terminal nucleotide or in 2′-5′ linked oligonucleotides and the 5′ position of 5′ terminal nucleotide. Modified sugars can also include those that contain modifications at the bridging ring oxygen, such as CH₂ and S. Nucleotide sugar analogs can also have sugar mimetics, such as cyclobutyl moieties in place of the pentofuranosyl sugar.

Nucleotide analogs can also be modified at the phosphate moiety. Modified phosphate moieties include, but are not limited to, those that can be modified so that the linkage between two nucleotides contains a phosphorothioate, chiral phosphorothioate, phosphorodithioate, phosphotriester, aminoalkylphosphotriester, methyl and other alkyl phosphonates including 3′-alkylene phosphonate and chiral phosphonates, phosphinates, phosphoramidates including 3′-amino phosphoramidate and aminoalkylphosphoramidates, thionophosphoramidates, thionoalkylphosphonates, thionoalkylphosphotriesters, and boranophosphates. These phosphate or modified phosphate linkage between two nucleotides can be through a 3′-5′ linkage or a 2′-5′ linkage, and the linkage can contain inverted polarity such as 3′-5′ to 5′-3′ or 2′-5′ to 5′-2′. Various salts, mixed salts, and free acid forms are also included. Nucleotide substitutes also include peptide nucleic acids (PNAs).

The present disclosure also provides vectors comprising any one or more of the nucleic acid molecules disclosed herein. In some embodiments, the vectors comprise any one or more of the nucleic acid molecules disclosed herein and a heterologous nucleic acid. The vectors can be viral or nonviral vectors capable of transporting a nucleic acid molecule. In some embodiments, the vector is a plasmid or cosmid (such as, for example, a circular double-stranded DNA into which additional DNA segments can be ligated). In some embodiments, the vector is a viral vector, wherein additional DNA segments can be ligated into the viral genome. Expression vectors include, but are not limited to, plasmids, cosmids, retroviruses, adenoviruses, adeno-associated viruses (AAV), plant viruses such as cauliflower mosaic virus and tobacco mosaic virus, yeast artificial chromosomes (YACs), Epstein-Barr (EBV)-derived episomes, and other expression vectors known in the art.

Desired regulatory sequences for mammalian host cell expression can include, for example, viral elements that direct high levels of polypeptide expression in mammalian cells, such as promoters and/or enhancers derived from retroviral LTRs, cytomegalovirus (CMV) (such as, for example, CMV promoter/enhancer), Simian Virus 40 (SV40) (such as, for example, SV40 promoter/enhancer), adenovirus, (such as, for example, the adenovirus major late promoter (AdMLP)), polyoma and strong mammalian promoters such as native immunoglobulin and actin promoters. Methods of expressing polypeptides in bacterial cells or fungal cells (such as, for example, yeast cells) are also well known. A promoter can be, for example, a constitutively active promoter, a conditional promoter, an inducible promoter, a temporally restricted promoter (such as, for example, a developmentally regulated promoter), or a spatially restricted promoter (such as, for example, a cell-specific or tissue-specific promoter).

Percent identity (or percent complementarity) between particular stretches of nucleotide sequences within nucleic acid molecules or amino acid sequences within polypeptides can be determined routinely using BLAST programs (basic local alignment search tools) and PowerBLAST programs (Altschul et al., J. Mol. Biol., 1990, 215, 403-410; Zhang and Madden, Genome Res., 1997, 7, 649-656) or by using the Gap program (Wisconsin Sequence Analysis Package, Version 8 for Unix, Genetics Computer Group, University Research Park, Madison Wis.), using default settings, which uses the algorithm of Smith and Waterman (Adv. Appl. Math., 1981, 2, 482-489). Herein, if reference is made to percent sequence identity, the higher percentages of sequence identity are preferred over the lower ones.

The present disclosure also provides compositions comprising any one or more of the isolated nucleic acid molecules, genomic nucleic acid molecules, mRNA molecules, and/or cDNA molecules disclosed herein. In some embodiments, the composition is a pharmaceutical composition. In some embodiments, the compositions comprise a carrier and/or excipient. Examples of carriers include, but are not limited to, poly(lactic acid) (PLA) microspheres, poly(D,L-lactic-coglycolic-acid) (PLGA) microspheres, liposomes, micelles, inverse micelles, lipid cochleates, and lipid microtubules. A carrier may comprise a buffered salt solution such as PBS, HBSS, etc.

As used herein, the phrase “corresponding to” or grammatical variations thereof when used in the context of the numbering of a particular nucleotide or nucleotide sequence or position refers to the numbering of a specified reference sequence when the particular nucleotide or nucleotide sequence is compared to a reference sequence (such as, for example, SEQ ID NO:1, SEQ ID NO:3, or SEQ ID NO:19). In other words, the residue (such as, for example, nucleotide or amino acid) number or residue (such as, for example, nucleotide or amino acid) position of a particular polymer is designated with respect to the reference sequence rather than by the actual numerical position of the residue within the particular nucleotide or nucleotide sequence. For example, a particular nucleotide sequence can be aligned to a reference sequence by introducing gaps to optimize residue matches between the two sequences. In these cases, although the gaps are present, the numbering of the residue in the particular nucleotide or nucleotide sequence is made with respect to the reference sequence to which it has been aligned.

For example, an ST6GALNAC5 missense nucleic acid molecule comprising a nucleotide sequence encoding an ST6GALNAC5 predicted loss-of-function polypeptide, wherein the nucleotide sequence comprises a cytosine at a position corresponding to position 176,867 according to SEQ ID NO:2 means that if the nucleotide sequence of the ST6GALNAC5 genomic nucleic acid molecule is aligned to the sequence of SEQ ID NO:2, the ST6GALNAC5 sequence has a cytosine residue at the position that corresponds to position 176,867 of SEQ ID NO:2. The same applies for an ST6GALNAC5 missense mRNA molecules comprising a nucleotide sequence encoding an ST6GALNAC5 predicted loss-of-function polypeptide, wherein the nucleotide sequence comprises a cytosine at a position corresponding to position 600 according to SEQ ID NO:11, and an ST6GALNAC5 missense cDNA molecules comprising a nucleotide sequence encoding an ST6GALNAC5 predicted loss-of-function polypeptide, wherein the nucleotide sequence comprises a cytosine at a position corresponding to position 600 according to SEQ ID NO:27. In other words, these phrases refer to a nucleic acid molecule encoding an ST6GALNAC5 polypeptide, wherein the genomic nucleic acid molecule has a nucleotide sequence that comprises a cytosine residue that is homologous to the cytosine residue at position 176,867 of SEQ ID NO:2 (or wherein the mRNA molecule has a nucleotide sequence that comprises a cytosine residue that is homologous to the cytosine residue at position 600 of SEQ ID NO:11, or wherein the cDNA molecule has a nucleotide sequence that comprises a cytosine residue that is homologous to the cytosine residue at position 600 of SEQ ID NO:27).

As described herein, a position within an ST6GALNAC5 missense genomic nucleic acid molecule that corresponds to position 176,867 according to SEQ ID NO:2, for example, can be identified by performing a sequence alignment between the nucleotide sequence of a particular ST6GALNAC5 nucleic acid molecule and the nucleotide sequence of SEQ ID NO:2. A variety of computational algorithms exist that can be used for performing a sequence alignment to identify a nucleotide position that corresponds to, for example, position 176,867 in SEQ ID NO:2. For example, by using the NCBI BLAST algorithm (Altschul et al., Nucleic Acids Res., 1997, 25, 3389-3402) or CLUSTALW software (Sievers and Higgins, Methods Mol. Biol., 2014, 1079, 105-116) sequence alignments may be performed. However, sequences can also be aligned manually.

The amino acid sequences of ST6GALNAC5 reference polypeptides are set forth in SEQ ID NO:35 (Isoform 1), SEQ ID NO:36 (Isoform 2), SEQ ID NO:37 (Isoform 3), and SEQ ID NO:38 (Isoform 4). Referring to SEQ ID NO:35 (Isoform 1), the ST6GALNAC5 reference polypeptide is 336 amino acids in length. Referring to SEQ ID NO:35, position 135 is a valine. Referring to SEQ ID NO:36 (Isoform 2), the ST6GALNAC5 reference polypeptide is 146 amino acids in length. Referring to SEQ ID NO:36, position 45 is a valine. Referring to SEQ ID NO:37 (Isoform 3), the ST6GALNAC5 reference polypeptide is 246 amino acids in length. Referring to SEQ ID NO:37, position 45 is a valine. Referring to SEQ ID NO:38 (Isoform 4), the ST6GALNAC5 reference polypeptide is 236 amino acids in length. Referring to SEQ ID NO:38, position 135 is a valine.

The amino acid sequences of ST6GALNAC5 predicted loss-of-function polypeptides are set forth in SEQ ID NO:39 (Isoform 1; Val135A1a), SEQ ID NO:40 (Isoform 2; Val45A1a), SEQ ID NO:41 (Isoform 3; Val45A1a*), and SEQ ID NO:42 (Isoform 4; Val135A1a*). Referring to SEQ ID NO:39, position 135 is an alanine. Referring to SEQ ID NO:40, position 45 is an alanine. Referring to SEQ ID NO:41, position 45 is an alanine. Referring to SEQ ID NO:42, position 135 is an alanine.

The nucleotide and amino acid sequences listed in the accompanying sequence listing are shown using standard letter abbreviations for nucleotide bases, and three-letter code for amino acids. The nucleotide sequences follow the standard convention of beginning at the 5′ end of the sequence and proceeding forward (i.e., from left to right in each line) to the 3′ end. Only one strand of each nucleotide sequence is shown, but the complementary strand is understood to be included by any reference to the displayed strand. The amino acid sequence follows the standard convention of beginning at the amino terminus of the sequence and proceeding forward (i.e., from left to right in each line) to the carboxy terminus.

The present disclosure also provides therapeutic agents that treat or prevent cognitive impairment for use in the treatment of cognitive impairment (or for use in the preparation of a medicament for treating cognitive impairment) in a subject, wherein the subject has any of the ST6GALNAC5 missense variant genomic nucleic acid molecules, missense variant mRNA molecules, and/or missense variant cDNA molecules encoding an ST6GALNAC5 predicted loss-of-function polypeptide described herein. The therapeutic agents that treat or prevent cognitive impairment can be any of the therapeutic agents that treat or prevent cognitive impairment described herein.

In some embodiments, the subject is identified as having a genomic nucleic acid molecule encoding an ST6GALNAC5 predicted loss-of-function polypeptide, wherein the genomic nucleic acid molecule has a nucleotide sequence comprising a cytosine at a position corresponding to position 176,867 according to SEQ ID NO:2, or the complement thereof.

In some embodiments, the subject is identified as having an mRNA molecule encoding an ST6GALNAC5 predicted loss-of-function polypeptide, wherein the mRNA molecule has a nucleotide sequence comprising: a cytosine at a position corresponding to position 600 according to SEQ ID NO:11, or the complement thereof; a cytosine at a position corresponding to position 639 according to SEQ ID NO:12, or the complement thereof; a cytosine at a position corresponding to position 562 according to SEQ ID NO:13, or the complement thereof; a cytosine at a position corresponding to position 515 according to SEQ ID NO:14, or the complement thereof; a cytosine at a position corresponding to position 579 according to SEQ ID NO:15, or the complement thereof; a cytosine at a position corresponding to position 416 according to SEQ ID NO:16, or the complement thereof; a cytosine at a position corresponding to position 639 according to SEQ ID NO:17, or the complement thereof; or a cytosine at a position corresponding to position 600 according to SEQ ID NO:18, or the complement thereof.

In some embodiments, the subject is identified as having a cDNA molecule encoding an ST6GALNAC5 predicted loss-of-function polypeptide, wherein the cDNA molecule has a nucleotide sequence comprising: a cytosine at a position corresponding to position 600 according to SEQ ID NO:27, or the complement thereof; a cytosine at a position corresponding to position 639 according to SEQ ID NO:28, or the complement thereof; a cytosine at a position corresponding to position 562 according to SEQ ID NO:29, or the complement thereof; a cytosine at a position corresponding to position 515 according to SEQ ID NO:30, or the complement thereof; a cytosine at a position corresponding to position 579 according to SEQ ID NO:31, or the complement thereof; a cytosine at a position corresponding to position 416 according to SEQ ID NO:32, or the complement thereof; a cytosine at a position corresponding to position 639 according to SEQ ID NO:33, or the complement thereof; or a cytosine at a position corresponding to position 600 according to SEQ ID NO:34, or the complement thereof.

In some embodiments, the subject is identified as having: i) a genomic nucleic acid molecule having a nucleotide sequence encoding an ST6GALNAC5 predicted loss-of-function polypeptide, wherein the nucleotide sequence comprises a cytosine at a position corresponding to position 176,867 according to SEQ ID NO:2, or the complement thereof; ii) an mRNA molecule having a nucleotide sequence encoding an ST6GALNAC5 predicted loss-of-function polypeptide, wherein the nucleotide sequence comprises: a cytosine at a position corresponding to position 600 according to SEQ ID NO:11, or the complement thereof; a cytosine at a position corresponding to position 639 according to SEQ ID NO:12, or the complement thereof; a cytosine at a position corresponding to position 562 according to SEQ ID NO:13, or the complement thereof; a cytosine at a position corresponding to position 515 according to SEQ ID NO:14, or the complement thereof; a cytosine at a position corresponding to position 579 according to SEQ ID NO:15, or the complement thereof; a cytosine at a position corresponding to position 416 according to SEQ ID NO:16, or the complement thereof; a cytosine at a position corresponding to position 639 according to SEQ ID NO:17, or the complement thereof; or a cytosine at a position corresponding to position 600 according to SEQ ID NO:18, or the complement thereof; or iii) a cDNA molecule having a nucleotide sequence encoding an ST6GALNAC5 predicted loss-of-function polypeptide, wherein the nucleotide sequence comprises: a cytosine at a position corresponding to position 600 according to SEQ ID NO:27, or the complement thereof; a cytosine at a position corresponding to position 639 according to SEQ ID NO:28, or the complement thereof; a cytosine at a position corresponding to position 562 according to SEQ ID NO:29, or the complement thereof; a cytosine at a position corresponding to position 515 according to SEQ ID NO:30, or the complement thereof; a cytosine at a position corresponding to position 579 according to SEQ ID NO:31, or the complement thereof; a cytosine at a position corresponding to position 416 according to SEQ ID NO:32, or the complement thereof; a cytosine at a position corresponding to position 639 according to SEQ ID NO:33, or the complement thereof; or a cytosine at a position corresponding to position 600 according to SEQ ID NO:34, or the complement thereof.

In some embodiments, the subject is identified as having a genomic nucleic acid molecule having a nucleotide sequence encoding an ST6GALNAC5 predicted loss-of-function polypeptide, wherein the nucleotide sequence comprises a cytosine at a position corresponding to position 176,867 according to SEQ ID NO:2, or the complement thereof.

In some embodiments, the subject is identified as having an mRNA molecule having a nucleotide sequence encoding an ST6GALNAC5 predicted loss-of-function polypeptide, wherein the nucleotide sequence comprises: a cytosine at a position corresponding to position 600 according to SEQ ID NO:11, or the complement thereof; a cytosine at a position corresponding to position 639 according to SEQ ID NO:12, or the complement thereof; a cytosine at a position corresponding to position 562 according to SEQ ID NO:13, or the complement thereof; a cytosine at a position corresponding to position 515 according to SEQ ID NO:14, or the complement thereof; a cytosine at a position corresponding to position 579 according to SEQ ID NO:15, or the complement thereof; a cytosine at a position corresponding to position 416 according to SEQ ID NO:16, or the complement thereof; a cytosine at a position corresponding to position 639 according to SEQ ID NO:17, or the complement thereof; or a cytosine at a position corresponding to position 600 according to SEQ ID NO:18, or the complement thereof.

In some embodiments, the subject is identified as having a cDNA molecule having a nucleotide sequence encoding an ST6GALNAC5 predicted loss-of-function polypeptide, wherein the nucleotide sequence comprises: a cytosine at a position corresponding to position 600 according to SEQ ID NO:27, or the complement thereof; a cytosine at a position corresponding to position 639 according to SEQ ID NO:28, or the complement thereof; a cytosine at a position corresponding to position 562 according to SEQ ID NO:29, or the complement thereof; a cytosine at a position corresponding to position 515 according to SEQ ID NO:30, or the complement thereof; a cytosine at a position corresponding to position 579 according to SEQ ID NO:31, or the complement thereof; a cytosine at a position corresponding to position 416 according to SEQ ID NO:32, or the complement thereof; a cytosine at a position corresponding to position 639 according to SEQ ID NO:33, or the complement thereof; or a cytosine at a position corresponding to position 600 according to SEQ ID NO:34, or the complement thereof.

In some embodiments, the subject is identified as having an ST6GALNAC5 predicted loss-of-function polypeptide that comprises: an alanine at a position corresponding to position 135 according to SEQ ID NO:39, an alanine at a position corresponding to position 45 according to SEQ ID NO:40, an alanine at a position corresponding to position 45 according to SEQ ID NO:41, or an alanine at a position corresponding to position 135 according to SEQ ID NO:42.

The present disclosure also provides ST6GALNAC5 inhibitors for use in the treatment of cognitive impairment (or for use in the preparation of a medicament for treating cognitive impairment) in a subject, wherein the subject is heterozygous for any of the ST6GALNAC5 missense variant genomic nucleic acid molecules, missense variant mRNA molecules, and/or missense variant cDNA molecules encoding an ST6GALNAC5 predicted loss-of-function polypeptide described herein, or wherein the subject is reference for an ST6GALNAC5 genomic nucleic acid molecule, mRNA molecule, or cDNA molecule. The ST6GALNAC5 inhibitors can be any of the ST6GALNAC5 inhibitors described herein.

In some embodiments, the subject is reference for an ST6GALNAC5 genomic nucleic acid molecule, an ST6GALNAC5 mRNA molecule, or an ST6GALNAC5 cDNA molecule. In some embodiments, the subject is reference for an ST6GALNAC5 genomic nucleic acid molecule. In some embodiments, the subject is reference for an ST6GALNAC5 mRNA molecule. In some embodiments, the subject is reference for an ST6GALNAC5 cDNA molecule.

In some embodiments, the subject is identified as being heterozygous for a genomic nucleic acid molecule encoding an ST6GALNAC5 predicted loss-of-function polypeptide, wherein the genomic nucleic acid molecule has a nucleotide sequence comprising a cytosine at a position corresponding to position 176,867 according to SEQ ID NO:2, or the complement thereof.

In some embodiments, the subject is identified as being heterozygous for an mRNA molecule encoding an ST6GALNAC5 predicted loss-of-function polypeptide, wherein the mRNA molecule has a nucleotide sequence comprising: a cytosine at a position corresponding to position 600 according to SEQ ID NO:11, or the complement thereof; a cytosine at a position corresponding to position 639 according to SEQ ID NO:12, or the complement thereof; a cytosine at a position corresponding to position 562 according to SEQ ID NO:13, or the complement thereof; a cytosine at a position corresponding to position 515 according to SEQ ID NO:14, or the complement thereof; a cytosine at a position corresponding to position 579 according to SEQ ID NO:15, or the complement thereof; a cytosine at a position corresponding to position 416 according to SEQ ID NO:16, or the complement thereof; a cytosine at a position corresponding to position 639 according to SEQ ID NO:17, or the complement thereof; or a cytosine at a position corresponding to position 600 according to SEQ ID NO:18, or the complement thereof.

In some embodiments, the subject is identified as being heterozygous for a cDNA molecule encoding an ST6GALNAC5 predicted loss-of-function polypeptide, wherein the cDNA molecule has a nucleotide sequence comprising: a cytosine at a position corresponding to position 600 according to SEQ ID NO:27, or the complement thereof; a cytosine at a position corresponding to position 639 according to SEQ ID NO:28, or the complement thereof; a cytosine at a position corresponding to position 562 according to SEQ ID NO:29, or the complement thereof; a cytosine at a position corresponding to position 515 according to SEQ ID NO:30, or the complement thereof; a cytosine at a position corresponding to position 579 according to SEQ ID NO:31, or the complement thereof; a cytosine at a position corresponding to position 416 according to SEQ ID NO:32, or the complement thereof; a cytosine at a position corresponding to position 639 according to SEQ ID NO:33, or the complement thereof; or a cytosine at a position corresponding to position 600 according to SEQ ID NO:34, or the complement thereof.

In some embodiments, the subject is identified as being heterozygous for: i) a genomic nucleic acid molecule having a nucleotide sequence encoding an ST6GALNAC5 predicted loss-of-function polypeptide, wherein the nucleotide sequence comprises a cytosine at a position corresponding to position 176,867 according to SEQ ID NO:2, or the complement thereof; ii) an mRNA molecule having a nucleotide sequence encoding an ST6GALNAC5 predicted loss-of-function polypeptide, wherein the nucleotide sequence comprises: a cytosine at a position corresponding to position 600 according to SEQ ID NO:11, or the complement thereof; a cytosine at a position corresponding to position 639 according to SEQ ID NO:12, or the complement thereof; a cytosine at a position corresponding to position 562 according to SEQ ID NO:13, or the complement thereof; a cytosine at a position corresponding to position 515 according to SEQ ID NO:14, or the complement thereof; a cytosine at a position corresponding to position 579 according to SEQ ID NO:15, or the complement thereof; a cytosine at a position corresponding to position 416 according to SEQ ID NO:16, or the complement thereof; a cytosine at a position corresponding to position 639 according to SEQ ID NO:17, or the complement thereof; or a cytosine at a position corresponding to position 600 according to SEQ ID NO:18, or the complement thereof; or iii) a cDNA molecule having a nucleotide sequence encoding an ST6GALNAC5 predicted loss-of-function polypeptide, wherein the nucleotide sequence comprises: a cytosine at a position corresponding to position 600 according to SEQ ID NO:27, or the complement thereof; a cytosine at a position corresponding to position 639 according to SEQ ID NO:28, or the complement thereof; a cytosine at a position corresponding to position 562 according to SEQ ID NO:29, or the complement thereof; a cytosine at a position corresponding to position 515 according to SEQ ID NO:30, or the complement thereof; a cytosine at a position corresponding to position 579 according to SEQ ID NO:31, or the complement thereof; a cytosine at a position corresponding to position 416 according to SEQ ID NO:32, or the complement thereof; a cytosine at a position corresponding to position 639 according to SEQ ID NO:33, or the complement thereof; or a cytosine at a position corresponding to position 600 according to SEQ ID NO:34, or the complement thereof.

In some embodiments, the subject is identified as being heterozygous for a genomic nucleic acid molecule having a nucleotide sequence encoding an ST6GALNAC5 predicted loss-of-function polypeptide, wherein the nucleotide sequence comprises a cytosine at a position corresponding to position 176,867 according to SEQ ID NO:2, or the complement thereof.

In some embodiments, the subject is identified as being heterozygous for an mRNA molecule having a nucleotide sequence encoding an ST6GALNAC5 predicted loss-of-function polypeptide, wherein the nucleotide sequence comprises: a cytosine at a position corresponding to position 600 according to SEQ ID NO:11, or the complement thereof; a cytosine at a position corresponding to position 639 according to SEQ ID NO:12, or the complement thereof; a cytosine at a position corresponding to position 562 according to SEQ ID NO:13, or the complement thereof; a cytosine at a position corresponding to position 515 according to SEQ ID NO:14, or the complement thereof; a cytosine at a position corresponding to position 579 according to SEQ ID NO:15, or the complement thereof; a cytosine at a position corresponding to position 416 according to SEQ ID NO:16, or the complement thereof; a cytosine at a position corresponding to position 639 according to SEQ ID NO:17, or the complement thereof; or a cytosine at a position corresponding to position 600 according to SEQ ID NO:18, or the complement thereof.

In some embodiments, the subject is identified as being heterozygous for a cDNA molecule having a nucleotide sequence encoding an ST6GALNAC5 predicted loss-of-function polypeptide, wherein the nucleotide sequence comprises: a cytosine at a position corresponding to position 600 according to SEQ ID NO:27, or the complement thereof; a cytosine at a position corresponding to position 639 according to SEQ ID NO:28, or the complement thereof; a cytosine at a position corresponding to position 562 according to SEQ ID NO:29, or the complement thereof; a cytosine at a position corresponding to position 515 according to SEQ ID NO:30, or the complement thereof; a cytosine at a position corresponding to position 579 according to SEQ ID NO:31, or the complement thereof; a cytosine at a position corresponding to position 416 according to SEQ ID NO:32, or the complement thereof; a cytosine at a position corresponding to position 639 according to SEQ ID NO:33, or the complement thereof; or a cytosine at a position corresponding to position 600 according to SEQ ID NO:34, or the complement thereof.

All patent documents, websites, other publications, accession numbers and the like cited above or below are incorporated by reference in their entirety for all purposes to the same extent as if each individual item were specifically and individually indicated to be so incorporated by reference. If different versions of a sequence are associated with an accession number at different times, the version associated with the accession number at the effective filing date of this application is meant. The effective filing date means the earlier of the actual filing date or filing date of a priority application referring to the accession number if applicable. Likewise, if different versions of a publication, website or the like are published at different times, the version most recently published at the effective filing date of the application is meant unless otherwise indicated. Any feature, step, element, embodiment, or aspect of the present disclosure can be used in combination with any other feature, step, element, embodiment, or aspect unless specifically indicated otherwise. Although the present disclosure has been described in some detail by way of illustration and example for purposes of clarity and understanding, it will be apparent that certain changes and modifications may be practiced within the scope of the appended claims.

The following examples are provided to describe the embodiments in greater detail. They are intended to illustrate, not to limit, the claimed embodiments. The following examples provide those of ordinary skill in the art with a disclosure and description of how the compounds, compositions, articles, devices and/or methods described herein are made and evaluated, and are intended to be purely exemplary and are not intended to limit the scope of any claims. Efforts have been made to ensure accuracy with respect to numbers (such as, for example, amounts, temperature, etc.), but some errors and deviations may be accounted for. Unless indicated otherwise, parts are parts by weight, temperature is in ° C. or is at ambient temperature, and pressure is at or near atmospheric.

### EXAMPLES

### Example 1: Rare Variant Associations with Brain Imaging Traits

The exomes of 454,787 UKB study participants were sequenced, with 95.8% of targeted bases covered at a depth of 20× or greater, as previously described (Szustakowski, Advancing Human Genetics Research and Drug Discovery through Exome Sequencing of the UK Biobank. bioRxiv, 2021; and Van Hout et al., Nature, 2020). Twelve million variants were identified in 39 million base pairs across the coding regions of 18,659 genes (data not shown). Among the variants identified were 3,375,252 (median of 10,260 per individual) synonymous 7,689,495 (9,284 per individual) missense and 889,957 (212 per individual) putative loss-of-function (pLOF) variants (data not shown), of which about half were observed only once in this dataset (singleton variants; data not shown).

An imaging component of the UK Biobank was analyzed that included 36,968 individuals. An exome-wide association analyses was carried out on 2,077 brain imaging phenotypes derived from magnetic resonance imaging (MRI), conditional upon GWAS signals, to disaggregate the rare variant associations from the common variant signals. Overall, 343 associations with 52 genes were discovered at a P≤10⁻⁷, and 98 associations with seven genes at a more conservative significance threshold of P≤10⁻¹⁰ (data not shown). The strongest trait-increasing association with GWC was with a deleterious missense variant in ST6GALNAC5 (r5756654226, 9 carriers; effect=1.7 SD units, 95% Cl 1.1 to 2.4, P=8.2×10⁻⁸). This aligns with current evidence that the relative abundance of specific gangliosides in the brain changes with age and in common neurological conditions, including Huntington's disease, Alzheimer's disease, Parkinson's disease, amyotrophic lateral sclerosis, stroke, multiple sclerosis and epilepsy.

Various modifications of the described subject matter, in addition to those described herein, will be apparent to those skilled in the art from the foregoing description. Such modifications are also intended to fall within the scope of the appended claims. Each reference (including, but not limited to, journal articles, U.S. and non-U.S. patents, patent application publications, international patent application publications, gene bank accession numbers, and the like) cited in the present application is incorporated herein by reference in its entirety and for all purposes.

1. A method of treating a subject having cognitive impairment, decreased myelin integrity, neurodegeneration, decreased grey/white matter contrast (GWC), conversion of mild cognitive impairment to dementia or at risk of developing cognitive impairment, decreased myelin integrity, neurodegeneration, decreased GWC, or conversion of mild cognitive impairment to dementia, the method comprising administering an Alpha-N-Acetylgalactosaminide Alpha-2,6-Sialyltransferase 5 (ST6GALNAC5) inhibitor to the subject.

2-5. (canceled)

6. The method according to claim 1, wherein the ST6GALNAC5 inhibitor comprises an inhibitory nucleic acid molecule.

7. The method according to claim 6, wherein the inhibitory nucleic acid molecule comprises an antisense nucleic acid molecule, a small interfering RNA (siRNA), or a short hairpin RNA (shRNA) that hybridizes to an ST6GALNAC5 nucleic acid molecule.

8-14. (canceled)

15. The method according to claim 1, further comprising detecting the presence or absence of an ST6GALNAC5 missense variant nucleic acid molecule encoding an ST6GALNAC5 predicted loss-of-function polypeptide in a biological sample obtained from the subject.

16. The method according to claim 15, further comprising administering a therapeutic agent that treats or inhibits cognitive impairment in a standard dosage amount to a subject wherein the ST6GALNAC5 missense variant nucleic acid molecule is absent from the biological sample.

17. The method according to claim 15, further comprising administering a therapeutic agent that treats or inhibits cognitive impairment in a dosage amount that is the same as or less than a standard dosage amount to a subject that is heterozygous for the ST6GALNAC5 missense variant nucleic acid molecule.

18. The method according to claim 15, wherein the ST6GALNAC5 missense variant nucleic acid molecule encodes Val135Ala, Val45Ala, Val45A1a*, or Val135A1a*.

19. The method according to claim 15, wherein the ST6GALNAC5 missense variant nucleic acid molecule encodes Val135Ala, Val45Ala, Val45A1a*, or Val135A1a*.

20. The method according to claim 18, wherein the ST6GALNAC5 missense variant nucleic acid molecule is:
a genomic nucleic acid molecule having a nucleotide sequence comprising a cytosine at a position corresponding to position 176,867 according to SEQ ID NO:2;
an mRNA molecule having a nucleotide sequence comprising: a cytosine at a position corresponding to position 600 according to SEQ ID NO:11; a cytosine at a position corresponding to position 639 according to SEQ ID NO:12; a cytosine at a position corresponding to position 562 according to SEQ ID NO:13; a cytosine at a position corresponding to position 515 according to SEQ ID NO:14; a cytosine at a position corresponding to position 579 according to SEQ ID NO:15; a cytosine at a position corresponding to position 416 according to SEQ ID NO:16; a cytosine at a position corresponding to position 639 according to SEQ ID NO:17; or a cytosine at a position corresponding to position 600 according to SEQ ID NO:18; or
a cDNA molecule having a nucleotide sequence comprising: a cytosine at a position corresponding to position 600 according to SEQ ID NO:27; a cytosine at a position corresponding to position 639 according to SEQ ID NO:28; a cytosine at a position corresponding to position 562 according to SEQ ID NO:29; a cytosine at a position corresponding to position 515 according to SEQ ID NO:30; a cytosine at a position corresponding to position 579 according to SEQ ID NO:31; a cytosine at a position corresponding to position 416 according to SEQ ID NO:32; a cytosine at a position corresponding to position 639 according to SEQ ID NO:33; or a cytosine at a position corresponding to position 600 according to SEQ ID NO:34.

21-35. (canceled)

36. A method of treating a subject with a therapeutic agent that treats or prevents cognitive impairment, wherein the subject has cognitive impairment or is at risk of developing cognitive impairment, the method comprising:
determining whether the subject has an Alpha-N-Acetylgalactosaminide Alpha-2,6-Sialyltransferase 5 (ST6GALNAC5) missense variant nucleic acid molecule encoding an ST6GALNAC5 predicted loss-of-function polypeptide by:
obtaining or having obtained a biological sample from the subject; and
performing or having performed a sequence analysis on the biological sample to determine if the subject has a genotype comprising the ST6GALNAC5 missense variant nucleic acid molecule encoding the ST6GALNAC5 predicted loss-of-function polypeptide; and
administering or continuing to administer the therapeutic agent that treats or inhibits cognitive impairment in a standard dosage amount to a subject that is ST6GALNAC5 reference, and/or administering an ST6GALNAC5 inhibitor to the subject; and
administering or continuing to administer the therapeutic agent that treats or inhibits cognitive impairment in an amount that is the same as or less than a standard dosage amount to a subject that is heterozygous for the ST6GALNAC5 missense variant nucleic acid molecule, and/or administering an ST6GALNAC5 inhibitor to the subject;
wherein the presence of a genotype having the ST6GALNAC5 missense variant nucleic acid molecule encoding the ST6GALNAC5 predicted loss-of-function polypeptide indicates the subject has a reduced risk of developing cognitive impairment.

37. The method according to claim 36, wherein the subject is ST6GALNAC5 reference, and the subject is administered or continued to be administered the therapeutic agent that treats or inhibits cognitive impairment in a standard dosage amount, and is administered an ST6GALNAC5 inhibitor.

38. The method according to claim 36, wherein the subject is heterozygous for an ST6GALNAC5 missense variant nucleic acid molecule, and the subject is administered or continued to be administered the therapeutic agent that treats or inhibits cognitive impairment in an amount that is the same as or less than a standard dosage amount, and is administered an ST6GALNAC5 inhibitor.

39. The method according to claim 36, wherein the ST6GALNAC5 missense variant nucleic acid molecule encodes Val135Ala, Val45Ala, Val45A1a*, or Val135A1a*.

40. The method according to claim 36, wherein the ST6GALNAC5 missense variant nucleic acid molecule encodes Val135Ala, Val45Ala, Val45A1a*, or Val135A1a*.

41. The method according to claim 39, wherein the ST6GALNAC5 missense variant nucleic acid molecule is:
a genomic nucleic acid molecule having a nucleotide sequence comprising a cytosine at a position corresponding to position 176,867 according to SEQ ID NO:2;
an mRNA molecule having a nucleotide sequence comprising: a cytosine at a position corresponding to position 600 according to SEQ ID NO:11, a cytosine at a position corresponding to position 639 according to SEQ ID NO:12, a cytosine at a position corresponding to position 562 according to SEQ ID NO:13, a cytosine at a position corresponding to position 515 according to SEQ ID NO:14, a cytosine at a position corresponding to position 579 according to SEQ ID NO:15, a cytosine at a position corresponding to position 416 according to SEQ ID NO:16, a cytosine at a position corresponding to position 639 according to SEQ ID NO:17, or a cytosine at a position corresponding to position 600 according to SEQ ID NO:18; or
a cDNA molecule produced from an mRNA molecule, wherein the cDNA molecule has a nucleotide sequence comprising: a cytosine at a position corresponding to position 600 according to SEQ ID NO:27, a cytosine at a position corresponding to position 639 according to SEQ ID NO:28, a cytosine at a position corresponding to position 562 according to SEQ ID NO:29, a cytosine at a position corresponding to position 515 according to SEQ ID NO:30, a cytosine at a position corresponding to position 579 according to SEQ ID NO:31, a cytosine at a position corresponding to position 416 according to SEQ ID NO:32, a cytosine at a position corresponding to position 639 according to SEQ ID NO:33, or a cytosine at a position corresponding to position 600 according to SEQ ID NO:34.

42-56. (canceled)

57. The method according to claim 36, wherein the ST6GALNAC5 inhibitor comprises an inhibitory nucleic acid molecule.

58. The method according to claim 57, wherein the inhibitory nucleic acid molecule comprises an antisense nucleic acid molecule, a small interfering RNA (siRNA), or a short hairpin RNA (shRNA) that hybridizes to an ST6GALNAC5 nucleic acid molecule.

59-97. (canceled)
